SEQUENCE LISTING (10 sequences)

SEQ ID NO: 1 - protein, 2099 aa, Homo sapiens
MATDDKTSPTLDSANDLPRSPTSPSHLTHFKPLTPDQDEPPFKSAYSSFVNLFRFNKERAEGGQGEQQPLSGSWTSPQLPSRTQSVRSPTPYKKQLNEELQRRSSALDTRRKAEPTFGGHDPRTAVQLRSLSTVLKRLKEIMEGKSQDSDLKQYWMPDSQCKECYDCSEKFTTFRRRHHCRLCGQIFCSRCCNQEIPGKFMGYTGDLRACTYCRKIALSYAHSTDSNSIGEDLNALSDSACSVSVLDPSEPRTPVGSRKASRNIFLEDDLAWQSLIHPDSSNTPLSTRLVSVQEDAGKSPARNRSASITNLSLDRSGSPMVPSYETSVSPQANRTYVRTETTEDERKILLDSVQLKDLWKKICHHSSGMEFQDHRYWLRTHPNCIVGKELVNWLIRNGHIATRAQAIAIGQAMVDGRWLDCVSHHDQLFRDEYALYRPLQSTEFSETPSPDSDSVNSVEGHSEPSWFKDIKFDDSDTEQIAEEGDDNLANSASPSKRTSVSSFQSTVDSDSAASISLNVELDNVNFHIKKPSKYPHVPPHPADQKEYLISDTGGQQLSISDAFIKESLFNRRVEEKSKELPFTPLGWHHNNLELLREENGEKQAMERLLSANHNHMMALLQQLLHSDSLSSSWRDIIVSLVCQVVQTVRPDVKNQDDDMDIRQFVHIKKIPGGKKFDSVVVNGFVCTKNIAHKKMNSCIKNPKILLLKCSIEYLYREETKFTCIDPIVLQEREFLKNYVQRIVDVRPTLVLVEKTVSRIAQDMLLEHGITLVINVKSQVLERISRMTQGDLVMSMDQLLTKPHLGTCHKFYMQIFQLPNEQTKTLMFFEGCPQHLGCTIKLRGGSDYELARVKEILIFMICVAYHSQLEISFLMDEFAMPPTLMQNPSFHSLIEGRGHEGAVQEQYGGGSIPWDPDIPPESLPCDDSSLLESRIVFEKGEQENKNLPQAVASVKHQEHSTTACPAGLPCAFFAPVPESLLPLPVDDQQDALGSELPESLQQTVVLQDPKSQIRAFRDPLQDDTGLYVTEEVTSSEDKRKTYSLAFKQELKDVILCISPVITFREPFLLTEKGMRCSTRDYFAEQVYWSPLLNKEFKEMENRRKKQLLRDLSGLQGMNGSIQAKSIQVLPSHELVSTRIAEHLGDSQSLGRMLADYRARGGRIQPKNSDPFAHSKDASSTSSGKSGSKNEGDEERGLILSDAVWSTKVDCLNPINHQRLCVLFSSSSAQSSNAPSACVSPWIVTMEFYGKNDLTLGIFLERYCFRPSYQCPSMFCDTPMVHHIRRFVHGQGCVQIILKELDSPVPGYQHTILTYSWCRICKQVTPVVALSNESWSMSFAKYLELRFYGHQYTRRANAEPCGHSIHHDYHQYFSYNQMVASFSYSPIRLLEVCVPLPKIFIKRQAPLKVSLLQDLKDFFQKVSQVYVAIDERLASLKTDTFSKTREEKMEDIFAQKEMEEGEFKNWIEKMQARLMSSSVDTPQQLQSVFESLIAKKQSLCEVLQAWNNRLRDLFQQEKGRKRPSVPPSPGRLRQGEESKISAMDASPRNISPGLQNGEKEDRFLTTLSSQSSTSSTHLQLPTPPEVMSEQSVGGPPELDTASSSEDVFDGHLLGSTDSQVKEKSTMKAIFANLLPGNSYNPIPFPFDPDKHYLMYEHERVPIAVCEKEPSSIIAFALSCKEYRNALEELSKATQWNSAEEGLPTNSTSDSRPKSSSPIRLPEMSGGQTNRTTETEPQPTKKASGMLSFFRGTAGKSPDLSSQKRETLRGADSAYYQVGQTGKEGTENQGVEPQDEVDGGDTQKKQLINPHVELQFSDANAKFYCRLYYAGEFHKMREVILDSSEEDFIRSLSHSSPWQARGGKSGAAFYATEDDRFILKQMPRLEVQSFLDFAPHYFNYITNAVQQKRPTALAKILGVYRIGYKNSQNNTEKKLDLLVMENLFYGRKMAQVFDLKGSLRNRNVKTDTGKESCDVVLLDENLLKMVRDNPLYIRSHSKAVLRTSIHSDSHFLSSHLIIDYSLLVGRDDTSNELVVGIIDYIRTFTWDKKLEMVVKSTGILGGQGKMPTVVSPELYRTRFCEAMDKYFLMVPDHWTGLGLNCZ

SEQ ID NO: 2 - DNA, 6297 bp, Homo sapiens
atggccacagatgataagacgtccccaacactggactctgctaatgatttgcctcgatctcctactagtccttctcatctcacacactttaaacctttgactcctgatcaagatgagcccccttttaaatcagcttatagttcttttgtaaatctctttcgttttaacaaagagagagcagaaggaggccagggagaacagcagcctttgagtggaagttggaccagccctcagctcccttcgaggacacagtctgttaggtcacccacaccttataaaaagcagcttaatgaggaactccagcggcgctcttcagcattagacacaagaaggaaagcagaacctacctttggaggtcatgaccctcgtacagctgttcagcttcgaagcctcagcacagtattaaaacgcctcaaggaaatcatggaggggaaaagccaggatagtgacctgaaacaatactggatgccagatagccaatgtaaagagtgctatgactgtagtgagaaatttacaacctttaggcgcagacaccattgccgactatgtgggcagattttctgcagtcgttgctgtaatcaagaaatccctggaaaatttatgggctatacaggagacctccgagcttgcacatattgtagaaaaatagccttaagttatgctcattccacagacagtaattctattggggaagacttgaatgctctttcagattctgcttgctctgtgtctgtgcttgatccaagtgaaccccgaacacctgttgggagtaggaaagccagccgtaacatatttttagaggatgatttggcctggcaaagtttgattcatccagattcctcaaatactcctctttcaacaagacttgtatctgtgcaagaggatgctgggaaatctcctgctcgaaatagatcagccagcattactaacctgtcactggatagatctggttctcctatggtaccttcatatgagacatctgtcagtccccaggctaaccgaacatatgttaggacagagaccactgaggatgaacgcaaaattcttctggacagtgtgcagttaaaagacctgtggaaaaaaatctgccatcacagcagtggaatggagtttcaggatcaccgctactggttgagaacgcatcccaactgcattgtaggaaaggaattagtcaactggctaatccgaaatgggcatattgccacaagggcacaagctatagcaattggacaagcaatggttgatggacgttggctggattgtgttagtcatcacgaccagcttttcagagatgagtatgcgctgtatagaccactgcagagtacagaattttctgagacgccttctcccgacagtgactcagtgaactccgtggaaggacactctgagccatcctggtttaaagacataaagtttgatgacagtgacacagaacagatagctgaagaaggtgacgataatttggctaattctgccagtcctagcaagcgcacatcagtcagcagtttccagtccacagtggacagtgactcagccgcttctatcagcctgaacgtggagctggacaacgtgaacttccatatcaagaagccctccaagtacccacatgtgccccctcaccctgctgaccaaaaagagtatttgatttctgacactggaggacaacagctctcaataagtgacgctttcatcaaagaatccttatttaatcgccgagtagaggaaaaatccaaagagctgcctttcacacctttgggctggcatcataacaacctggagctcctgagggaggagaatggggagaaacaagccatggagaggttgctttcagctaatcataaccacatgatggcactactccagcagttgctccatagtgactcactgtcatcatcttggagggacatcatcgtgtcattggtctgccaggttgttcagacagtccgacctgatgtcaagaaccaggatgatgacatggatatccgtcagtttgtccacatcaaaaaaatcccaggtggaaagaagtttgattctgtggttgtcaatggctttgtttgtaccaagaacattgcacataaaaagatgaattcttgtattaaaaaccctaaaattcttctgttgaagtgttccattgagtatctctacagagaagaaactaagtttacttgcattgatcctattgtgcttcaggaaagggaattcttgaagaattatgtccagcgaatagttgatgttcgacccaccttggttcttgttgagaaaacagtgtctcggattgcccaggacatgttattggaacatggcattactttggtcattaatgtaaagtcacaagttttggaacgaatcagtcgaatgacccaaggtgatttagtgatgtcaatggaccagctgcttacgaaaccacacctgggcacttgtcacaaattttatatgcagatatttcagttgcctaatgaacaaaccaagacactgatgttttttgaaggttgtccacagcacctaggctgtacaatcaagctaagaggaggctctgattatgagctggctcgagttaaggagatcctaatatttatgatctgtgttgcttatcattctcaactagaaatatcctttctcatggatgaatttgctatgcctcccacattaatgcaaaacccttcattccattccctgattgagggacgagggcatgagggggctgtccaagagcagtacggtggaggttccatcccctgggatcctgacatccctcctgagtctctgccctgtgatgatagcagtttgctggaatcgaggattgtgtttgagaagggtgagcaggaaaataaaaatcttccgcaggctgttgcctctgtgaagcatcaagaacatagcacaacagcttgcccggcgggtctcccttgtgctttctttgcacctgtaccggaatcattgttgccactccctgtggatgaccaacaagatgctttaggcagcgagctgccagagagtttgcagcaaacagttgtgctgcaggatcccaaaagccagataagagcctttagagaccctctacaggatgacactggattatatgttactgaggaagtcacctcctctgaagataaacgaaagacttattctttggcctttaagcaggaattaaaagatgtgatcctctgtatctccccagtaatcacattccgagaaccctttcttttaactgaaaaggggatgagatgctctacccgagattattttgcagagcaggtttactggtctcctctcctcaataaagaattcaaagaaatggagaacaggaggaagaaacagctgctcagggatctctctggacttcagggcatgaatggaagtattcaggccaagtctattcaagtcttaccctcacatgagctagtgagcactagaattgctgagcatctgggcgatagccagagcttgggtagaatgctggccgattatcgagccagaggaggaagaattcagcccaaaaattcagacccttttgctcattcaaaggatgcatcaagtacttcaagtggcaaatcaggaagcaaaaacgagggtgatgaagagagagggcttattctgagtgatgctgtgtggtcaacaaaggtggactgtctgaatcccattaatcaccagagactttgtgtgctcttcagcagctcttctgcccagtccagcaatgctcctagtgcctgtgtcagtccttggattgtaacaatggaattttatggaaagaatgatcttacattaggaatatttttagagagatactgtttcaggccttcttatcagtgtccaagcatgttctgtgatacccccatggtacatcatattcggcgctttgttcatggccaaggctgtgtgcagataatcctgaaggagttggattctccagtacctggatatcagcatacaattcttacatattcctggtgtagaatctgcaaacaggtaacaccagttgttgctctttccaatgagtcctggtctatgtcatttgcaaaataccttgaacttaggttttatgggcaccagtatactcgcagagccaacgctgagccctgtggtcactccatccatcatgattatcaccagtatttctcctataaccagatggtggcgtctttcagttattctcccattcggcttcttgaagtatgtgttccactccccaaaatattcattaagcgtcaggccccattaaaagtgtcccttcttcaggatctgaaggacttctttcaaaaagtttcacaggtatatgttgccattgatgaaagacttgcatctttgaaaactgatacatttagtaaaacaagagaggaaaaaatggaagatatttttgcacagaaagagatggaagaaggtgagttcaagaactggattgagaagatgcaagcaaggctcatgtcttcctctgtagatacccctcagcaactgcagtcggtctttgagtcactcattgccaagaaacaaagtctctgtgaagtgctgcaagcttggaataacaggttgcgggaccttttccaacaggaaaagggtagaaagagaccttcagttcctccaagtcctggaagactgagacaaggggaagaaagcaagataagtgcgatggatgcatctccacggaatatttctccaggacttcagaatggagaaaaagaggatcgcttcttaacaactttgtccagccagagctccaccagttctactcatctccaattgcctacgccacctgaagtcatgtctgaacagtcagtgggagggccccctgagctagatacagccagcagttccgaagatgtgtttgatgggcatttgctgggatccacagacagccaagtgaaggaaaagtcaaccatgaaagccatctttgcaaatttgcttccaggaaatagctataatcctattccatttccttttgatccagataaacactacttaatgtatgaacatgaacgagtgcccattgcagtctgcgagaaggaacccagctccatcattgcttttgctctcagttgtaaagaataccgaaatgccttagaggaattgtctaaagcgactcagtggaacagtgccgaagaagggcttccaacaaatagtacttcagatagcagaccaaagagtagcagccctatcagattacctgaaatgagtggaggacagacaaatcgtacaacagaaacagaaccacaaccaaccaaaaaggcttctggaatgttgtccttcttcagagggacagcagggaaaagccccgatctctcgtcccagaagagagagaccttacgtggagcagatagtgcttactaccaggttgggcagacaggcaaggaggggaccgagaatcaaggcgttgagcctcaagatgaagtagatggaggagatacgcaaaagaagcaactcataaatcctcatgtggaacttcaattttcagatgctaatgccaagttttactgtcggctctactatgcgggagagtttcataagatgcgtgaagtgattctggacagcagtgaggaagatttcattcgttccctctcccactcatcaccctggcaggcccggggaggcaaatcaggagctgccttctatgcaactgaggatgatagatttattttgaagcaaatgcctcgtctggaagtccagtccttcctcgactttgcaccacattacttcaattatattacaaatgctgttcaacaaaagaggcccacggcgttggccaaaattcttggagtttacagaattggttataagaactctcagaacaacactgagaagaagttagatctccttgtcatggaaaatcttttctacgggagaaagatggcacaggtttttgatttgaagggctctcttaggaatcggaatgtaaaaactgacactggaaaagagagttgtgatgtggtcctgctagatgaaaatctcctaaagatggttcgagacaaccctctatatattcgttctcattccaaagctgtgctgagaacctcgatccatagtgactcccatttcctttctagccacctcattatagattattctttgctggttgggcgagatgatactagcaatgagctagtagttggaattatagattatattcgaacatttacatgggacaaaaagcttgagatggttgtgaaatcaacaggaattttaggtggacaaggtaaaatgccaacagtggtgtctccggagttgtacaggactaggttttgtgaggcaatggacaagtatttcctaatggtaccagaccactggacaggcttgggtctgaattgctga

SEQ ID NO: 3 - protein, 907 aa, Homo sapiens
MPTAAAPIISSVQKLVLYETRARYFLVGSNNAETKYRVLKIDRTEPKDLVIIDDRHVYTQQEVRELLGRLDLGNRTKMGQKGSSGLFRAVSAFGVVGFVRFLEGYYIVLITKRRKMADIGGHAIYKVEDTNMIYIPNDSVRVTHPDEARYLRIFQNVDLSSNFYFSYSYDLSHSLQYNLTVLRMPLEMLKSEMTQNRQESFDIFEDEGLITQGGSGVFGICSEPYMKYVWNGELLDIIKSTVHRDWLLYIIHGFCGQSKLLIYGRPVYVTLIARRSSKFAGTRFLKRGANCEGDVANEVETEQILCDASVMSFTAGSYSSYVQVRGSVPLYWSQDISTMMPKPPITLDQADPFAHVAALHFDQMFQRFGSPIIILNLVKEREKRKHERILSEELVAAVTYLNQFLPPEHTIVYIPWDMAKYTKSKLCNVLDRLNVIAESVVKKTGFFVNRPDSYCSILRPDEKWNELGGCVIPTGRLQTGILRTNCVDCLDRTNTAQFMVGKCALAYQLYSLGLIDKPNLQFDTDAVRLFEELYEDHGDTLSLQYGGSQLVHRVKTYRKIAPWTQHSKDIMQTLSRYYSNAFSDADRQDSINLFLGVFHPTEGKPHLWELPTDFYLHHKNTMRLLPTRRSYTYWWTPEVIKHLPLPYDEVICAANLKKLIVKKFHKYEEEIDIHNEFFRPYELSSFDDTFCLAMTSSARDFMPKTVGIDPSPFTVRKPDETGKSVLGNKSNREEAVLQRKTAASAPPPPSEEAVSSSSEDDSGTDREEEGSVSQRSTPVKMTDAGDSAKVTENVVQPMKELYGINLSDGLSEEDFSIYSRFVQLGQSQHKQDKNSQQPCSRCSDGVIKLTPISAFSQDNIYEVQPPRVDRKSTEIFQAHIQASQGIMQPLGKEDSSMYREYIRNRYL

SEQ ID NO: 4 - DNA, 2724 bp, Homo sapiens
atgcccacggccgccgcccccatcatcagctcggtccagaagctggttctgtatgagactagagctagatactttctagttgggagcaataatgcagaaacgaaatatcgtgtcttgaagattgatagaacagaaccaaaagatttggtcataattgatgacaggcatgtctatactcaacaagaagtaagggaacttcttggccgcttggatcttggaaatagaacaaagatgggacagaaaggatcctcgggcttatttcgagcggtttcagcttttggtgttgtgggttttgtcaggttcttagaaggctattatattgtgttaataactaaaaggaggaagatggcggatattggaggtcatgcaatctataaggtcgaagatacaaatatgatctatatacccaatgattctgtacgggttactcatcctgatgaagctaggtatctacgaatatttcaaaatgtggacctatctagcaatttttactttagttacagctatgatttgtcccactcacttcaatataatctcactgtcttgcgaatgcccctggagatgttaaagtcagaaatgacccagaatcgccaagagagctttgacatctttgaagatgaaggattaattacacaaggtggaagcggggtatttgggatctgtagtgagccttatatgaaatatgtatggaatggtgaacttctggatataattaaaagtactgtgcatcgtgactggcttttgtatattattcatgggttctgtgggcagtcaaagctgttgatctatggacgaccagtgtatgtcactctaatagctagaagatccagtaaatttgctggcacccgttttcttaaaagaggtgcaaactgtgagggtgatgttgcaaatgaagtggagactgaacaaatactctgcgatgcttctgtgatgtctttcactgcaggaagttattcttcatatgtacaagttagaggatctgtgcccttatactggtctcaggacatttcaactatgatgcctaaaccacctattacattggatcaggcagatccatttgcacatgtggctgcccttcactttgaccagatgttccagaggtttggctctcccatcatcatcttgaatttagtgaaggaacgagagaaaagaaagcatgaaagaattctgagtgaagaacttgttgctgctgtgacctatctcaaccaatttttgcctcctgagcacactattgtttatattccctgggacatggccaagtataccaaaagcaagctgtgtaatgttcttgatcgactaaatgtgattgcagaaagtgtggtgaagaaaacaggtttctttgtaaaccgccctgattcttactgtagcattttgcggccagatgaaaagtggaatgaactaggaggatgtgtgattcccactggtcgcctgcagactggcatccttcgaaccaactgtgtggactgtttagatcgcaccaacacagcacagtttatggtgggaaaatgtgctctggcctatcagctgtattcactgggactgattgacaaacctaatctacagtttgatacagatgcagttaggttatttgaggaactctatgaagatcatggtgataccctatcccttcagtatggtggttctcaacttgttcatcgtgtgaaaacctacagaaagatagcaccatggacccagcactccaaagacatcatgcaaaccctgtctagatattacagcaatgctttttcagatgccgatagacaagattccattaatctcttcctgggagttttccatcccactgaagggaaacctcatctctgggagctcccaacagatttttatttgcatcacaaaaataccatgagacttttgccaacaagaagaagttatacttactggtggacaccagaggtgataaagcatttaccattgccctatgatgaagttatctgtgctgcgaacttaaagaagttgatagtgaagaaattccacaaatatgaagaagagattgatatccacaatgagttctttcggccatatgagttgagcagctttgatgataccttttgcttggctatgacaagctcagcacgtgactttatgcctaagaccgttggaattgatccaagtccatttactgtgcgtaaaccagatgaaactggaaaatcagtattgggaaacaaaagcaatagagaagaagctgtattacagcggaaaacggcagccagcgccccgccgccccccagcgaggaggctgtgtccagcagctctgaggatgactctgggactgatcgggaagaagagggctctgtgtctcagcgctccactcccgtgaagatgactgatgcaggagacagtgccaaagtgaccgagaatgtggtccaacccatgaaggagctatatggaattaacctctcagatggcctctcagaagaagatttctccatttattcaagatttgttcagctggggcagagtcaacataaacaagacaagaatagccagcagccctgttctaggtgctcagatggagttataaaactaacacccatctcggctttctcgcaagataacatctatgaagttcagcccccaagagtagacagaaaatctacagagatcttccaagcccacatccaggccagccaaggtatcatgcagcccctaggaaaagaggactcctccatgtaccgagagtacatcaggaaccgctacctgtga

SEQ ID NO: 5 - protein, 782 aa, Homo sapiens
MNPEKDFAPLTPNIVRALNDKLYEKRKVAALEIEKLVREFVAQNNTVQIKHVIQTLSQEFALSQHPHSRKGGLIGLAACSIALGKDSGLYLKELIEPALTCFNDADSRLRYYACEALYNIVKVARGAVLPHFNVLFDGLSKLAADPDPNVKSGSELLDRLLKDIVTESNKFDLVSFIPLLRERIYSNNQYARQFIISWILVLESVPDINLLDYLPEILDGLFQILGDNGKEIRKMCEVVLGEFLKEIKKNPSSVKFAEMANILVIHCQTTDDLIQLTAMCWMREFIQLAGRVMLPYSSGILTAVLPCLAYDDRKKSIKEVANVCNQSLMKLVTPEDDELDELRPGQRQAEPTPDDALPKQEGTASGGPDGSCDSSFSSGISVFTAASTERAPVTLHLDGIVQVLNCHLSDTAIGMMTRIAVLKWLYHLYIKTPRKMFRHTDSLFPILLQTLSDESDEVILKDLEVLAEIASSPAGQTDDPGPLDGPDLQASHSELQVPTPGRAGLLNTSGTKGLECSPSTPTMNSYFYKFMINLLKRFSSERKLLEVRGPFIIRQLCLLLNAENIFHSMADILLREEDLKFASTMVHALNTILLTSTELFQLRNQLKDLKTLESQNLFCCLYRSWCHNPVTTVSLCFLTQNYRHAYDLIQKFGDLEVTVDFLAEVDKLVQLIECPIFTYVRLQLLDVKNNPYLIKALYGLLMLLPQSSAFQLLSHRLQCVPNPELLQTEDSLKAAPKSQKADSPSIDYAELLQHFEKVQNKHLEVRHQRSGRGDHLDRRVVL

SEQ ID NO: 6 - DNA, 2349 bp, Homo sapiens
atgaaccccgagaaggatttcgcgccgctcacgcctaacatcgtgcgcgccctcaatgacaagctgtacgaaaagcggaaggtggcagcgctggagatcgagaagctggtccgggagttcgtggcccagaacaataccgtgcaaatcaagcatgtgatccagaccctgtcccaggagtttgccctgtctcagcacccccacagccggaaagggggcctcatcggcctggccgcctgctccatcgcactgggcaaggactcagggctctacctgaaggagctgatcgagccagctctgacctgcttcaatgatgcagacagcaggctgcgctactatgcctgcgaggccctctacaacatcgtcaaggtggcccggggcgctgtgctgccccacttcaacgtgctctttgacgggctgagcaagctggcagccgacccagaccccaatgtgaaaagcggatctgagctcctagaccgccttttaaaggacattgtgactgagagcaacaagtttgacctggtgagcttcatccccttgttgcgagagaggatttactccaacaaccagtatgcccggcagttcatcatctcctggatcctggttctggagtcggtgccagacattaacctgctggattacctgccggagatcctggatggactcttccagatcctgggtgacaatggcaaagagattcgcaaaatgtgtgaggttgttcttggagaattcttaaaagaaattaagaagaacccctccagtgtgaagtttgctgagatggccaacatcctggtgatccactgccagacaacagatgacctcatccagctgacagccatgtgctggatgcgggagttcatccagctggcgggccgcgtcatgctgccttactcctccgggatcctgactgctgtcttgccctgcttggcctacgatgaccgcaagaaaagcatcaaagaagtggccaacgtgtgcaaccagagcctgatgaagctggtcacccccgaggacgacgagctggatgagctgagacctgggcagaggcaggcagagcccacccctgacgatgccctgccaaagcaggagggcacagccagtggaggtccagatggttcctgtgactccagcttcagtagcggcatcagtgtcttcactgcagccagcactgaaagagccccagtgacccttcacctcgacgggatcgtgcaggtcctaaactgccacctcagtgacacggccattgggatgatgaccaggattgcagttctcaagtggctctaccacctctacatcaaaactcctcggaagatgttccggcacacggacagcctctttcccatcctactgcagacgttatcggatgaatcggatgaggtgatcctgaaggacctggaggtgctggcagaaatcgcttcctcccccgcaggccagacggatgacccaggccccctcgatggccctgacctccaggccagccactcagagctccaggtgcccacccctggcagagccggcctactgaacacctctggtaccaaaggcttagaatgttctccttcaactcccaccatgaattcttacttttataagttcatgatcaaccttctcaagagattcagcagcgaacggaagctcctggaggtcagaggccctttcatcatcaggcagctgtgcctcctgctgaatgcggagaacatcttccactcaatggcagacatcctgctgcgggaggaggacctcaagttcgcctcgaccatggtccacgccctcaacaccatcctgctgacctccacagagctcttccagctaaggaaccagctgaaggacctgaagaccctggagagccagaacctgttctgctgcctgtaccgctcctggtgccacaacccagtcaccacggtgtccctctgcttcctcacccagaactaccggcacgcctatgacctcatccagaagtttggggacctggaggtcaccgtggacttcctcgcagaggtggacaagctggtgcagctgattgagtgccccatcttcacatatgtgcgcctgcagctgctggacgtgaagaacaacccctacctgatcaaggccctctacggcctgctcatgctcctgccgcagagcagcgccttccagctgctctcgcaccggctccagtgcgtgcccaaccctgagctgctgcagaccgaagacagtctaaaggcagcccccaagtcccagaaagctgactcccctagcatcgactacgcagagctgctgcagcactttgagaaggtccagaacaagcacctggaagtgcggcaccagcggagcgggcgtggggaccacctggaccggagggttgtcctctga

SEQ ID NO: 7 - protein, 880 aa, Vac14 Yeast
MEKSIAKGLSDKLYEKRKAAALELEKLVKQCVLEGDYDRIDKIIDELCRDYAYALHQPMARNAGLMGLAATAIALGINDVGRYLRNILPPVLACFGDQNDQVRFYACESLYNIAKIAKGEILVYFNEIFDVLCKISADTENSVRGAAELLDRLIKDIVAERASNYISIVNNGSHGLLPAIKTDPISGDVYQEEYEQDNQLAFSLPKFIPLLTERIYAINPDTRVFLVDWLKVLLNTPGLELISYLPSFLGGLFTFLGDSHKDVRTVTHTLMDSLLHEVDRISKLQTEIKMKRLERLKMLEDKYNNSSTPTKKADGALIAEKKKTLMTALGGLSKPLSMETDDTKLSNTNETDDERHLTSQEQLLDSEATSQEPLRDGEEYIPGQDINLNFPEVITVLVNNLASSEAEIQLIALHWIQVILSISPNVFIPFLSKILSVLLKLLSDSDPHITEIAQLVNGQLLSLCSSYVGKETDGKIAYGPIVNSLTLQFFDSRIDAKIACLDWLILIYHKAPNQILKHNDSMFLTLLKSLSNRDSVLIEKALSLLQSLCSDSNDNYLRQFLQDLLTLFKRDTKLVKTRANFIMRQISSRLSPERVYKVISSILDNYNDTTFVKMMIQILSTNLITSPEMSSLRNKLRTCEDGMFFNSLFKSWCPNPVSVISLCFVAENYELAYTVLQTYANYELKLNDLVQLDILIQLFESPVFTRMRLQLLEQQKHPFLHKCLFGILMIIPQSKAFETLNRRLNSLNIWTSQSYVMNNYIRQRENSNFCDSNSDISQRSVSQSKLHFQELINHFKAVSEEDEYSSDMIRLDHGANNKSLLLGSFLDGIDEDKQEIVTPISPMNEAINEEMESPNDNSSVILKDSGSLPFNRNVSDKLKK

SEQ ID NO: 8 - DNA, 2643 bp, Vac14 Yeast
atggaaaaatcgattgccaaaggtttaagcgacaaactatacgaaaaacggaaggctgccgccttggagttggaaaagttggtcaaacaatgcgttctagaaggggactatgatcgtattgacaagataatagacgaactatgcagagactatgcatacgctttgcatcagcccatggctagaaatgcaggattaatgggcttggctgccacagcaattgctttaggaataaacgatgttggccgttacttgaggaatattcttcctccagttctcgcatgttttggcgaccagaatgaccaagtaagattttatgcgtgtgagagtttgtacaatattgcaaagattgcgaagggtgaaatactggtctatttcaatgaaatatttgacgtactctgtaaaattagtgctgatacagaaaactcggtccgaggagcagctgaactattagatcggttgataaaagatattgttgctgaaagagcatccaattacataagcattgtaaataatggatcgcatggtttgcttccagccataaaaacggatccaatcagcggggatgtctaccaagaagaatatgagcaagataatcaattggcgttctctttaccaaaatttattcctttattaacagagagaatttacgccattaaccctgatacgcgtgtttttttagttgactggcttaaagtcttgctcaataccccaggactagagctcatatcgtatctaccatcatttcttggcgggttattcacgttcttgggcgattctcataaagatgtcagaacagttacacacacccttatggactcactacttcacgaagtcgaccgcatttcaaaattacaaactgaaattaaaatgaagagactggaaagactgaaaatgctggaagataaatacaataacagttctactccaacgaaaaaggccgatggtgcacttattgctgaaaagaagaaaactttaatgactgcattaggtgggttatccaaacctctgagtatggagactgatgataccaaattatctaatactaatgagacagatgatgaaagacacttgacaagccaggagcagctgctagatagcgaggcaactagccaagaacctcttcgcgatggcgaagaatatatacctggtcaagatatcaacctcaatttcccagaggttattaccgttcttgtgaacaatttggcatcatctgaggcagagattcaattgattgcattacactggattcaggttattttatccatttctccaaatgtgtttatccccttcctttcaaaaattctttctgtgcttttaaagctattgagcgattcggatccgcatattactgaaattgctcagctggtgaacggtcaattactctcattgtgcagctcatatgtgggaaaagagacagacgggaaaattgcatacgggccaatcgttaatagcttaactctacagtttttcgatagtaggattgatgccaagattgcctgcttggattggctcatcttaatttatcacaaggctccaaaccaaatcttgaaacataacgacagcatgttcctaactttgttgaagtcattatcaaatagagactcggtgctaatcgagaaagctttaagtctcttacagagtttatgttctgactcaaatgataattatttacgtcagtttcttcaagatttattaactttattcaagagagataccaaattggtgaaaactagagcaaactttatcatgagacagatatcttcccgtttatcaccagaacgtgtttataaagtaatttcttcgatattggacaattataacgacacaacgtttgtgaaaatgatgattcagattttaagtacaaacttaataacttcaccggagatgtcgtcgttaagaaacaaacttagaacttgtgaagacggtatgtttttcaatagtttattcaaatcttggtgtcccaaccccgtgtccgtgatatccttatgttttgtagcagaaaattatgagttagcttacacagtgttacaaacttacgcgaactacgagttgaagttaaatgatctagtgcagttggatattttgattcagttgttcgaatcacccgttttcacaaggatgcggttgcaacttttagaacaacagaaacatccgtttctgcataagtgtctatttggtatactgatgattataccacagtcaaaggcatttgaaacattgaaccgcagattgaatagtttgaacatatggacatcccaatcttatgtcatgaacaactatataagacaaagggagaatagtaacttttgtgattccaattctgatatctcgcaaaggtccgttagccaaagtaaactacatttccaggaactaattaatcattttaaggccgtttcagaagaagatgaatattcatcagatatgataaggcttgaccatggtgctaacaataaatcacttctgcttggaagttttttggacggtattgacgaagataaacaagaaattgtcacaccaatctcgcccatgaatgaagctattaacgaggagatggaatctcccaacgataatagttctgtcatattaaaagatagtggcagtctgccattcaaccgcaatgtatccgataaattaaaaaaataa

SEQ ID NO: 9 - protein, 2042 aa, Homo sapiens
MATDDKTSPTLDSANDLPRSPTSPSHLTHFKPLTPDQDEPPFKSAYSSFVNLFRFNKERAEGGQGEQQPLSGSWTSPQLPSRTQSVRSPTPYKKQLNEELQRRSSALDTRRKAEPTFGGHDPRTAVQLRSLSTVLKRLKEIMEGKSQDSDLKQYWMPDSQCKECYDCSEKFTTFRRRHHCRLCGQIFCSRCCNQEIPGKFMGYTGDLRACTYCRKIALSYAHSTDSNSIGEDLNALSDSACSVSVLDPSEPRTPVGSRKASRNIFLEDDLAWQSLIHPDSSNTPLSTRLVSVQEDAGKSPARNRSASITNLSLDRSGSPMVPSYETSVSPQANRTYVRTETTEDERKILLDSVQLKDLWKKICHHSSGMEFQDHRYWLRTHPNCIVGKELVNWLIRNGHIATRAQAIAIGQAMVDGRWLDCVSHHDQLFRDEYALYRPLQSTEFSETPSPDSDSVNSVEGHSEPSWFKDIKFDDSDTEQIAEEGDDNLAKYLISDTGGQQLSISDAFIKESLFNRRVEEKSKELPFTPLGWHHNNLELLREENGEKQAMERLLSANHNHMMALLQQLLHSDSLSSSWRDIIVSLVCQVVQTVRPDVKNQDDDMDIRQFVHIKKIPGGKKFDSVVVNGFVCTKNIAHKKMNSCIKNPKILLLKCSIEYLYREETKFTCIDPIVLQEREFLKNYVQRIVDVRPTLVLVEKTVSRIAQDMLLEHGITLVINVKSQVLERISRMTQGDLVMSMDQLLTKPHLGTCHKFYMQIFQLPNEQTKTLMFFEGCPQHLGCTIKLRGGSDYELARVKEILIFMICVAYHSQLEISFLMDEFAMPPTLMQNPSFHSLIEGRGHEGAVQEQYGGGSIPWDPDIPPESLPCDDSSLLESRIVFEKGEQENKNLPQAVASVKHQEHSTTACPAGLPCAFFAPVPESLLPLPVDDQQDALGSELPESLQQTVVLQDPKSQIRAFRDPLQDDTGLYVTEEVTSSEDKRKTYSLAFKQELKDVILCISPVITFREPFLLTEKGMRCSTRDYFAEQVYWSPLLNKEFKEMENRRKKQLLRDLSGLQGMNGSIQAKSIQVLPSHELVSTRIAEHLGDSQSLGRMLADYRARGGRIQPKNSDPFAHSKDASSTSSGKSGSKNEGDEERGLILSDAVWSTKVDCLNPINHQRLCVLFSSSSAQSSNAPSACVSPWIVTMEFYGKNDLTLGIFLERYCFRPSYQCPSMFCDTPMVHHIRRFVHGQGCVQIILKELDSPVPGYQHTILTYSWCRICKQVTPVVALSNESWSMSFAKYLELRFYGHQYTRRANAEPCGHSIHHDYHQYFSYNQMVASFSYSPIRLLEVCVPLPKIFIKRQAPLKVSLLQDLKDFFQKVSQVYVAIDERLASLKTDTFSKTREEKMEDIFAQKEMEEGEFKNWIEKMQARLMSSSVDTPQQLQSVFESLIAKKQSLCEVLQAWNNRLRDLFQQEKGRKRPSVPPSPGRLRQGEESKISAMDASPRNISPGLQNGEKEDRFLTTLSSQSSTSSTHLQLPTPPEVMSEQSVGGPPELDTASSSEDVFDGHLLGSTDSQVKEKSTMKAIFANLLPGNSYNPIPFPFDPDKHYLMYEHERVPIAVCEKEPSSIIAFALSCKEYRNALEELSKATQWNSAEEGLPTNSTSDSRPKSSSPIRLPEMSGGQTNRTTETEPQPTKKASGMLSFFRGTAGKSPDLSSQKRETLRGADSAYYQVGQTGKEGTENQGVEPQDEVDGGDTQKKQLINPHVELQFSDANAKFYCRLYYAGEFHKMREVILDSSEEDFIRSLSHSSPWQARGGKSGAAFYATEDDRFILKQMPRLEVQSFLDFAPHYFNYITNAVQQKRPTALAKILGVYRIGYKNSQNNTEKKLDLLVMENLFYGRKMAQVFDLKGSLRNRNVKTDTGKESCDVVLLDENLLKMVRDNPLYIRSHSKAVLRTSIHSDSHFLSSHLIIDYSLLVGRDDTSNELVVGIIDYIRTFTWDKKLEMVVKSTGILGGQGKMPTVVSPELYRTRFCEAMDKYFLMVPDHWTGLGLNC

SEQ ID NO: 10 - DNA, 6129 bp, Homo sapiens
atggccacagatgataagacgtccccaacactggactctgctaatgatttgcctcgatctcctactagtccttctcatctcacacactttaaacctttgactcctgatcaagatgagcccccttttaaatcagcttatagttcttttgtaaatctctttcgttttaacaaagagagagcagaaggaggccagggagaacagcagcctttgagtggaagttggaccagccctcagctcccttcgaggacacagtctgttaggtcacccacaccttataaaaagcagcttaatgaggaactccagcggcgctcttcagcattagacacaagaaggaaagcagaacctacctttggaggtcatgaccctcgtacagctgttcagcttcgaagcctcagcacagtattaaaacgcctcaaggaaatcatggaggggaaaagccaggatagtgacctgaaacaatactggatgccagatagccaatgtaaagagtgctatgactgtagtgagaaatttacaacctttaggcgcagacaccattgccgactatgtgggcagattttctgcagtcgttgctgtaatcaagaaatccctggaaaatttatgggctatacaggagacctccgagcttgcacatattgtagaaaaatagccttaagttatgctcattccacagacagtaattctattggggaagacttgaatgctctttcagattctgcttgctctgtgtctgtgcttgatccaagtgaaccccgaacacctgttgggagtaggaaagccagccgtaacatatttttagaggatgatttggcctggcaaagtttgattcatccagattcctcaaatactcctctttcaacaagacttgtatctgtgcaagaggatgctgggaaatctcctgctcgaaatagatcagccagcattactaacctgtcactggatagatctggttctcctatggtaccttcatatgagacatctgtcagtccccaggctaaccgaacatatgttaggacagagaccactgaggatgaacgcaaaattcttctggacagtgtgcagttaaaagacctgtggaaaaaaatctgccatcacagcagtggaatggagtttcaggatcaccgctactggttgagaacgcatcccaactgcattgtaggaaaggaattagtcaactggctaatccgaaatgggcatattgccacaagggcacaagctatagcaattggacaagcaatggttgatggacgttggctggattgtgttagtcatcacgaccagcttttcagagatgagtatgcgctgtatagaccactgcagagtacagaattttctgagacgccttctcccgacagtgactcagtgaactccgtggaaggacactctgagccatcctggtttaaagacataaagtttgatgacagtgacacagaacagatagctgaagaaggtgacgataatttggctaagtatttgatttctgacactggaggacaacagctctcaataagtgacgctttcatcaaagaatccttatttaatcgccgagtagaggaaaaatccaaagagctgcctttcacacctttgggctggcatcataacaacctggagctcctgagggaggagaatggggagaaacaagccatggagaggttgctttcagctaatcataaccacatgatggcactactccagcagttgctccatagtgactcactgtcatcatcttggagggacatcatcgtgtcattggtctgccaggttgttcagacagtccgacctgatgtcaagaaccaggatgatgacatggatatccgtcagtttgtccacatcaaaaaaatcccaggtggaaagaagtttgattctgtggttgtcaatggctttgtttgtaccaagaacattgcacataaaaagatgaattcttgtattaaaaaccctaaaattcttctgttgaagtgttccattgagtatctctacagagaagaaactaagtttacttgcattgatcctattgtgcttcaggaaagggaattcttgaagaattatgtccagcgaatagttgatgttcgacccaccttggttcttgttgagaaaacagtgtctcggattgcccaggacatgttattggaacatggcattactttggtcattaatgtaaagtcacaagttttggaacgaatcagtcgaatgacccaaggtgatttagtgatgtcaatggaccagctgcttacgaaaccacacctgggcacttgtcacaaattttatatgcagatatttcagttgcctaatgaacaaaccaagacactgatgttttttgaaggttgtccacagcacctaggctgtacaatcaagctaagaggaggctctgattatgagctggctcgagttaaggagatcctaatatttatgatctgtgttgcttatcattctcaactagaaatatcctttctcatggatgaatttgctatgcctcccacattaatgcaaaacccttcattccattccctgattgagggacgagggcatgagggggctgtccaagagcagtacggtggaggttccatcccctgggatcctgacatccctcctgagtctctgccctgtgatgatagcagtttgctggaatcgaggattgtgtttgagaagggtgagcaggaaaataaaaatcttccgcaggctgttgcctctgtgaagcatcaagaacatagcacaacagcttgcccggcgggtctcccttgtgctttctttgcacctgtaccggaatcattgttgccactccctgtggatgaccaacaagatgctttaggcagcgagctgccagagagtttgcagcaaacagttgtgctgcaggatcccaaaagccagataagagcctttagagaccctctacaggatgacactggattatatgttactgaggaagtcacctcctctgaagataaacgaaagacttattctttggcctttaagcaggaattaaaagatgtgatcctctgtatctccccagtaatcacattccgagaaccctttcttttaactgaaaaggggatgagatgctctacccgagattattttgcagagcaggtttactggtctcctctcctcaataaagaattcaaagaaatggagaacaggaggaagaaacagctgctcagggatctctctggacttcagggcatgaatggaagtattcaggccaagtctattcaagtcttaccctcacatgagctagtgagcactagaattgctgagcatctgggcgatagccagagcttgggtagaatgctggccgattatcgagccagaggaggaagaattcagcccaaaaattcagacccttttgctcattcaaaggatgcatcaagtacttcaagtggcaaatcaggaagcaaaaacgagggtgatgaagagagagggcttattctgagtgatgctgtgtggtcaacaaaggtggactgtctgaatcccattaatcaccagagactttgtgtgctcttcagcagctcttctgcccagtccagcaatgctcctagtgcctgtgtcagtccttggattgtaacaatggaattttatggaaagaatgatcttacattaggaatatttttagagagatactgtttcaggccttcttatcagtgtccaagcatgttctgtgatacccccatggtacatcatattcggcgctttgttcatggccaaggctgtgtgcagataatcctgaaggagttggattctccagtacctggatatcagcatacaattcttacatattcctggtgtagaatctgcaaacaggtaacaccagttgttgctctttccaatgagtcctggtctatgtcatttgcaaaataccttgaacttaggttttatgggcaccagtatactcgcagagccaacgctgagccctgtggtcactccatccatcatgattatcaccagtatttctcctataaccagatggtggcgtctttcagttattctcccattcggcttcttgaagtatgtgttccactccccaaaatattcattaagcgtcaggccccattaaaagtgtcccttcttcaggatctgaaggacttctttcaaaaagtttcacaggtatatgttgccattgatgaaagacttgcatctttgaaaactgatacatttagtaaaacaagagaggaaaaaatggaagatatttttgcacagaaagagatggaagaaggtgagttcaagaactggattgagaagatgcaagcaaggctcatgtcttcctctgtagatacccctcagcaactgcagtcggtctttgagtcactcattgccaagaaacaaagtctctgtgaagtgctgcaagcttggaataacaggttgcgggaccttttccaacaggaaaagggtagaaagagaccttcagttcctccaagtcctggaagactgagacaaggggaagaaagcaagataagtgcgatggatgcatctccacggaatatttctccaggacttcagaatggagaaaaagaggatcgcttcttaacaactttgtccagccagagctccaccagttctactcatctccaattgcctacgccacctgaagtcatgtctgaacagtcagtgggagggccccctgagctagatacagccagcagttccgaagatgtgtttgatgggcatttgctgggatccacagacagccaagtgaaggaaaagtcaaccatgaaagccatctttgcaaatttgcttccaggaaatagctataatcctattccatttccttttgatccagataaacactacttaatgtatgaacatgaacgagtgcccattgcagtctgcgagaaggaacccagctccatcattgcttttgctctcagttgtaaagaataccgaaatgccttagaggaattgtctaaagcgactcagtggaacagtgccgaagaagggcttccaacaaatagtacttcagatagcagaccaaagagtagcagccctatcagattacctgaaatgagtggaggacagacaaatcgtacaacagaaacagaaccacaaccaaccaaaaaggcttctggaatgttgtccttcttcagagggacagcagggaaaagccccgatctctcgtcccagaagagagagaccttacgtggagcagatagtgcttactaccaggttgggcagacaggcaaggaggggaccgagaatcaaggcgttgagcctcaagatgaagtagatggaggagatacgcaaaagaagcaactcataaatcctcatgtggaacttcaattttcagatgctaatgccaagttttactgtcggctctactatgcgggagagtttcataagatgcgtgaagtgattctggacagcagtgaggaagatttcattcgttccctctcccactcatcaccctggcaggcccggggaggcaaatcaggagctgccttctatgcaactgaggatgatagatttattttgaagcaaatgcctcgtctggaagtccagtccttcctcgactttgcaccacattacttcaattatattacaaatgctgttcaacaaaagaggcccacggcgttggccaaaattcttggagtttacagaattggttataagaactctcagaacaacactgagaagaagttagatctccttgtcatggaaaatcttttctacgggagaaagatggcacaggtttttgatttgaagggctctcttaggaatcggaatgtaaaaactgacactggaaaagagagttgtgatgtggtcctgctagatgaaaatctcctaaagatggttcgagacaaccctctatatattcgttctcattccaaagctgtgctgagaacctcgatccatagtgactcccatttcctttctagccacctcattatagattattctttgctggttgggcgagatgatactagcaatgagctagtagttggaattatagattatattcgaacatttacatgggacaaaaagcttgagatggttgtgaaatcaacaggaattttaggtggacaaggtaaaatgccaacagtggtgtctccggagttgtacaggactaggttttgtgaggcaatggacaagtatttcctaatggtaccagaccactggacaggcttgggtctgaattgctga

(19) United States
(12) Patent Application Publication
(10) Pub. No.: US 2005/0142561 A1
(43) Pub. Date: Jun. 30, 2005

(54) Intracellular signaling pathways in diabetic subjects

(76) Inventor: Lois Weisman, Iowa City, IA (US)

Correspondence Address: Steven L. Highlander;Fulbright & Jaworski L.L.P., Suite 2400, 600 Congress Avenue, AUSTIN, TX, 78701, US

(21) Appl. No.: 10/791,219

(22) Filed: Mar. 2, 2004

Related U.S. Application Data
(60) Provisional application No. 60/452,782, filed on Mar. 7, 2003.

(51) Int. Cl. C12Q 1/68; G01N 33/53; G01N 33/567; C07H 21/04; C07K 14/47; C07K 16/18

(52) U.S. Cl. 435/6; 435/7.2; 435/69.1; 435/320.1; 435/325; 530/350; 530/388.1; 536/23.5

(57) ABSTRACT

The present invention provides for methods of identifying subjects with a predisposition towards, or the existing condition of, type II diabetes. The methods involve examination of at least three human gene products—Fab1p, Vac14p and Fig4p—each of which play an important role in the insulin-response pathway. In addition, the invention provides methods for screening of potential therapeutic compounds, and methods for the treatment of type I and II diabetes.

This application claims the benefit of U.S. Provisional Application No. 60/452,782 filed Mar. 7, 2003. The U.S. Government owns rights in the present invention pursuant to grant number GM50403 and DK25295 from the National Institutes of Health.

### BACKGROUND OF THE INVENTION

1. Field of the Invention

The present invention relates generally to the field of human molecular biology and pathology, and more particularly to the area of diabetes. Specifically, it provides for the characterization of an intracellular signaling pathways that are involved in response to insulin. Compositions and methods are disclosed that provide for diagnostic or prognostic applications, and well as for the screening of potential drugs to treat diabetes.

2. Description of Related Art

β-cells of the islets of Langerhans in the pancreas secrete insulin in response to secretagogues such as amino acids, glyceraldehyde, free fatty acids; and, most prominently, glucose. Increased insulin secretion in response to a glucose load prevents hyperglycemia in normal individuals by stimulating glucose uptake into peripheral tissues, particularly muscle and adipose tissue. Individuals who lack proper insulin/glucose regulation suffer from diabetes.

Insulin-dependent diabetes mellitus, or IDDM (also known as Juvenile-onset or Type I diabetes), represents approximately 10% of all human diabetes. IDDM is distinct from non-insulin dependent diabetes (NIDDM) in that only IDDM involves specific destruction of the insulin producing β-cells of the islets of Langerhans. The destruction of α-cells in IDDM appears to be a result of specific autoimmune attack, in which the patient's own immune system recognizes and destroys the β-cells, but not the surrounding α-cells (glucagon producing) or 6-cells (somatostatin producing) that comprise the islet.

Type II diabetes, in contrast to type I, appears to arise at least in part from the inability of cells to respond to insulin. Insulin is responsible for stimulating glucose uptake into its target cells by a process which involves the translocation of the GLUT4 isoform of glucose transporter from an intracellular vesicular compartment(s) to the plasma membrane. Thus, despite an ability to sense glucose and send proper signals (insulin) for glucose uptake, the afflicted individuals nonetheless suffer from poor glucose clearance and storage. The pathways that are responsible for insulin response unfortunately remain obscure.

Thus, it is clear that there remains a critical need to identify the mechanism through which insulin-responsive cells receive and process insulin-based signals. In so doing, it will be possible to both identify individuals who have, or who are at risk of developing type II diabetes, and also screen for drugs that can modulate these pathways, thereby permitting treatment of both type I and II diabetes.

### SUMMARY OF THE INVENTION

The present invention overcomes these and other drawbacks inherent in the prior art by providing composition and methods of using the same in the diagnosis or prediction of diabetes. Also provided are methods for screening of drugs that can alter the insulin-response in insulin-responsive cells. Finally, methods of treating type I and type II diabetes are presented.

Thus, in one embodiment, there is provided an isolated and purified nucleic acid encoding a polypeptide comprising the sequence of SEQ ID NO:1, 3, 5 or 7. The isolated and purified nucleic acid may comprise the sequence of SEQ ID NO:2, 4, 6 or 8. 3. Expression constructs comprises these nucleic acids are also contemplated. In another embodiment, there is provided oligonucleotide comprising at least about 10 consecutive bases of SEQ ID NO:2, 4, 6 or 8. The oliogonucleotide may be 10, 15, 20, 25, 30, 35, 40, 45 or 50 bases in length, and the number of consecutive bases may be 10, 15, 20, 25, 30, 35, 40, 45 or 50 bases. In yet another embodiment, there is provided an expression cassette comprising a promoter active in eukaryotic cells, the promoter operably linked to a nucleic acid segment encoding a polypeptide comprising the sequence of SEQ ID NO:1, SEQ ID NO:3, SEQ ID NO:5 or SEQ ID NO:7, the promoter also being heterologous to the nucleic acid segment. The expression cassette may be comprised in an episomal element or integrated into the cellular genome.

In still yet another embodiment, there is provide an isolated and purified polypeptide comprising the sequence of SEQ ID NO:1, 3, 5 or 7. Also contemplated are fusion proteins involving each of these sequences. Other proteinaceous embodiments include (a) an oligopeptide of between about 5 and about 30 residues, the oligopeptide comprising at least about 5 consecutive residues of SEQ ID NO:1, SEQ ID NO:3, SEQ ID NO:5 or SEQ ID NO:7, where the oliogopeptide may be 5, 10, 15, 20, 25, or 30 residues in length, and where the number of consecutive residues may be 5, 10, 15, 20, 25, or 30; (b) a monoclonal antibody that binds immunologically to a polypeptide comprising the sequence of SEQ ID NO:1, 3, 5 or 7; and (c) a polyclonal antisera, antibodies of which bind immunologically to a polypeptide comprising the sequence of SEQ ID NO:1, 3, 5 or 7.

In a further embodiment, there is provided a non-human transgenic animal, cells of which comprise expression cassette comprising a promoter active in eukaryotic cells, the promoter operably linked to a nucleic acid segment encoding a polypeptide comprising the sequence of SEQ ID NO:1, SEQ ID NO:3, SEQ ID NO:5 or SEQ ID NO:7, the promoter also being heterologous to the nucleic acid segment. The animal's cells may further cells exhibit a defect in the expression of a Fab1, Vac14 or Fig4 polypeptide, such as reduced expression of Fab1, Vac14 or Fig4 polypeptide, or expression of a reduced-function or non-functional Fab1, Vac14 or Fig4 polypeptide.

In still a further embodiment, there is provided a method of identifying a subject at risk of developing diabetes comprising assessing the structure, function or expression of Fab1, Vac14 and/or Fig4 in cells of the subject. Assessing may comprise assessing expression, such as Northern blotting, quantitative RT-PCR, Western blotting or quantitative immunohistochemistry. Assessing may also comprise assessing activity, such as measuring PI(3,5)P₂, measuring PI(3,5)P₂ turnover, measuring PI(3,5)P₂ steady state levels, measuring PI(3,5)P₂ synthesis, measuring PI(3)P, or measuring protein kinase activity. Assessing may comprise assessing structure, such as (a) nucleic acid sequencing, including PCR- and RT-PCR-based studies, (b) measuring antibody binding, such as RIA, ELISA, Western blot or immunohistochemistry, and (c) high stringency nucleic acid hybridization. The method may further comprise obtaining a cell from the subject, such as a kidney cell, a liver cell, a leukocyte, an adipocyte, or a muscle cell. The method may further comprise subjecting the cell to stress prior to assessing expression or activity, such as osmotic stress. The method may further comprise subjecting the cell to hormonal stimulation prior to assessing expression or activity, such as insulin stimulation.

In an additional embodiment, there is provided a method of screening a candidate compound for their ability to increase glucose uptake comprising (a) providing a insulin-responsive cell; (b) contacting the insulin-responsive cells with the candidate compound; and (c) measuring the change in PI(3,5)P₂ in the cell. The insulin-responsive cell may be an adipocyte or a muscle cell.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Proteins with identity to Vac14p exist in higher eukaryotes. Identical amino acids (black) and similar amino acids (gray) are highlighted. (Left) The NH2-terminal sequence of S. cerevisiae VAC14 and related ORFs were aligned using ClustalW (searchlauncher.bcm.tmc.edu:9331/multi-align/multi-align.html). Sequences were identified by searching the indicated databases via the BLAST algorithm (Altschul et al., 1990). The C. albicans sequence was found in the C. albicans database (sequence-www.stanford.edu/group/candida/search.html). ORFs from S. pombe (EMBL/GenBank/DDBJ accession no. CAB08779.1), A. thaliana (EMBL/GenBank/DDBJ accession no. AAD12702.1), C. elegans (EMBL/GenBank/DDBJ accession no. CAB00043.1) and D. melanogaster (EMBL/GenBank/DDBJ accession no. AAF54829.1) were in the GenBank database (www.ncbi.nlm.nih.gov/BLAST). The M. musculus sequence was identified in the mouse EST database (www.ncbi.nlm.nih.gov/BLAST). The sequence shown is a consensus of two similar ESTs (EMBL/GenBank/DDBJ accession nos. BE573148 and BF162275). The H. sapiens sequence was in the human EST database (www.ncbi.nlm.nih.gov/BLAST). The consensus sequence of 14 similar ESTs from chromosome 16 (EMBL/GenBank/DDBJ accession nos. AL527155, AL535971, AL555680, AL556062, BE409891, BE696780, BE728471, BE893810, BE901196, BE937614, BF081182, BF091052, BF325708, and BG107035) is shown. The sequences contain at least 25% global identity and 42% global similarity to S. cerevisiae VAC14. (Right) The COOH-terminal sequence of yeast Vac14p and similar ORFs were identified and aligned as in the left sequence. The M. musculus sequence is a consensus of 12 similar ESTs (EMBL/GenBank/DDBJ accession nos. AA036005, AA050423, AA058300, AA276168, AA497446, AA670618, BE862623, BF023070, BF237130, BF720417, BG079707, W09660). The H. sapiens sequence is hypothetical protein, FLJ10305 found on chromosome 16, deposited in the human genome database (www.ncbi.nlm.nih.gov/genome/seq). One of the mouse ESTs (clone ID 468926) had been mapped to chromosome VIII (106 cM offset) with an inferred position on human chromosome 16 (16q22.1-qter).

FIG. 2—Alignment of Saccharomyces cerevisiae (Sc) Fig4p and human (Hs) Fig4p. Amino acid sequences were aligned using the ClustalW program. The aligned sequences were divided into groups of ten and assigned a color bar based on their percent identity. The absence of a colored bar indicates a gap introduced by the ClustalW program to optimize the alignment.

FIG. 3—The levels of PI3P and PI3,5P2 transiently change in response to hyperosmotic stress. Cells were labeled with myo-[2-3H]inositol for 12 hours and exposed to 0.9M NaCl for the indicated times. The dotted line indicates total PI3P+PI3,5P2.

FIG. 4—Fab1p is a large protein with several domains. The four identified domains are indicated by hatched boxes. The arrow indicates the single amino acid change in fab1-2.

FIG. 5—The steady state levels of PI3,5P2 are regulated via its rate of synthesis. Vps34p synthesizes PI3P which is converted to PI3,5P2 by Fab1p. Vac7p and Vac14p are required for Fab1p activation under all conditions. Vac14p may have a specialized role in osmotic stress, while Fig4p is only required during osmotic stress. The mechanism of Vps34p activation is unkinown. The enzymes required for turnover may also be regulated; first inactivated, and subsequently stimulated.

FIG. 6—Levels of PI3P and PI3,5P2 in the Class III mutant yeast strains after osmotic stress. Cells were labeled with myo-[2-3H]inositol for 12 hours and exposed to 0.9M NaCl for the indicated times. The lines shown in gray indicate PPI levels in a wild-type strain.

FIG. 7—Alignment of Saccharomyces cerevisiae Vac14p and human Vac14p. Amino acid sequences were aligned using the ClustalW program. The aligned sequences were divided into groups of ten and assigned a color bar based on their percent identity. The absence of a colored bar indicates a gap introduced by the ClustalW program to optimize the alignment.

FIG. 8—Possible isoforms of human Fab1p. The N-terminal portion of human Fab1p was amplified from a human adipocyte cDNA pool (Clontech) and cloned into a bacterial vector. 46 isolates were categorized by restriction digest and a representative of each category was sequenced. Another possible isoform, clone #BF981526 from small intestine, was found with a BLAST search of the human EST database. The FYVE domain, which binds PI3P, is indicated by a black box. Another variable region, exon 11, may be human specific and is indicated by a gray box.

### DESCRIPTION OF ILLUSTRATIVE EMBODIMENTS

In light of the need for alternative therapeutic strategies for treating diabetes, the present inventor has examined a signaling pathway present in insulin-responsive cells in an effort to determine how such cells normally respond to insulin, and what genetic defects might contribute to the lack of proper insulin response. As discussed in the following paragraphs, the inventor has determined that a recently discovered lipid, PI(3,5)P₂, plays a critical role in signaling pathways that are involved in insulin response. In addition, they have identified the human homologs to three yeast genes—Fab1p, Vac14p and Fig4p—which are important actors in this pathway.

The inventor and others have recently found that PI3,5P₂ exists at very low levels in unperturbed cells. In the inventor's work with yeast, it has been discovered that cells defective in PI3,5P₂ production display large, unlobed vacuoles (lysosomes) (Gary et al., 1998; Bonangelino et al., 1997) and are defective in retrograde traffic out of the vacuole (Bryant et al., 1998). Most notably, it has been shown that PI3,5P₂ increases 20-fold in response to hyperosmotic shock (Gary et al., 1998; Dove et al., 1997). The rapidity of this response is remarkable—upon transfer of cells to high salt media, full elevation of PI3,5P₂ levels occurs in just 5 minutes and returns to basal levels by 30 minutes (unpublished results). This dramatic increase is likely the result of Fab1p activation, and not inhibition of PI3,5P₂ turnover. This hypothesis is supported by observations that PIKfyve partially substitutes for yeast Fab1p (McEwen et al., 1999). However, it only restores basal levels of PI3,5P₂ and is not activated in response to salt shock. This loss of ability to elevate PI3,5P₂ is due to a defect in Fab1p/PIKfyve activation because all other cellular components are still present. These results strongly suggest that activation of Fab1p/PIKfyve provides the major contribution to the increase in PI3,5P₂.

Type 2, in contrast to type I diabetes, is characterized by blood glucose and insulin levels that are higher than normal due to the inability of cells to properly uptake glucose from the bloodstream. This defect is rooted in the signal transduction pathway that relays the external insulin stimulation to the internal mobilization of glucose transporters (GLUT4, GLUT1) to the plasma membrane. Recent results with a mammalian homologue of Fab1p, termed PIKfyve, closely parallel yeast studies and strongly suggest that PIKfyve, and its product PI3,5P₂, may be important in GLUT4 translocation.

In order to further explore this relationship further, the inventor developed a hypothesis based on previous studies which demonstrated that mouse PI4P 5-kinase (Itoh et al., 2000), mouse PI3P 5-kinase (PIKfyve) (Sbrissa et al., 2000) and S. pombe PI4P 5-kinase (Vancurova et al., 1999) have both lipid and protein kinase activities and are capable of autophosphorylation. Moreover, in vitro lipid kinase activity is dramatically increased following treatment with alkaline phosphatase. These studies suggest that Fab1p may be phosphorylated under basal conditions and then dephosphorylated during hyperosmotic shock or insulin stimulation, thereby increasing Fab1p activity. In support of this model, the inventor demonstrated that immunoprecipitated HA-Fab1p from ³²P-labeled yeast cells is phosphorylated under basal conditions, and further found that HA-Fab1p isolated from hyperosmotically shocked cells had a large decrease in phosphorylation, coinciding with the dramatic increase in PI3,5P₂ levels observed in these cells.

Vac14p and Vac7p are activators of Fab1p (Bonangelino et al., 1997), and the inventor has recently discovered that Fig4p is an activator as well (unpublished results). The corresponding knock-out strains, Δfig4, Δvac14 and Δvac7, like Δfab1, are defective in synthesis of PI3,5P₂. This defect results in abnormally enlarged vacuoles. Moreover, elevation of PI3,5P₂ levels in response to hyperosmotic shock is defective. Overexpression of FAB1 in these mutants partially suppresses the defect suggesting that Fig4p, Vac14p and Vac7p act upstream of Fab1p. Furthermore, Fig4p and Vac14p have been shown to interact in a yeast two-hybrid test (Dove et al., 2002; unpublished results). The precise functions of Vac14p and Vac7p, however, have not yet been elucidated. Each protein has no significant degree of homology with any other eukaryotic protein. However, Vac14p does have weak homology with PR65, a regulatory subunit of mammalian protein phosphatase 2A. Therefore, Vac14p may play a role in regulating the protein phosphatase that dephosphorylates Fab1p.

While a specific function for Fig4p has yet to be identified, sequence analysis suggests that it should function as a lipid phosphatase, since it contains a Sac1 PI phosphatase domain. Based on this observation, the inventor generated a Δfig4 strain to investigate the role of Fig4p in possible turnover of PI3,5P₂. Notably, they observed that the strain had enlarged vacuoles, a phenotype that is very similar to the Δvac14 strain. Moreover, Δfig4 cells do not produce elevated levels of PI3,5P₂ in response to hyperosmotic shock. These results are consistent with a hypothesis that Fig4p is an activator of PI3,5P₂ production. This surprising finding led the inventor to consider how this activation could occur.

An intriguing possibility is that Fig4p functions as a protein phosphatase and contributes directly, or indirectly, to the dephosphorylation of Fab1p. Support for a predicted PI lipid phosphatase functioning as a protein phosphatase comes from the observation that mammalian PTEN and myotubularin, members of the protein tyrosine phosphatase superfamily, are in fact lipid phosphatases (Maehama and Dixon, 1998; Taylor et al., 2000). This suggests that the sequences for these two types of phosphatases can be similar. In addition, some of these proteins may have dual phosphatase activities. Likewise, the predicted lipid phosphatase Fig4p may have protein phosphatase activity, as outlined above, and lipid phosphatase activity, as detailed in Gary et al. (2002), and outlined in Example 1. The inventor found that PI3,5P₂ in Δfig4 cells only increases 4-fold after hyperosmotic shock, supporting a role for Fig4p in the activation of Fab1p. Notably, these same levels persist past the 30 minute time point. This is in stark contrast to wild-type cells where a 20-fold increase in PI3,5P₂ is followed by a return to basal levels by 30 minutes. This prolonged elevation of PI3,5P₂ levels in Δfig4 suggests that both synthesis and turnover of PI3,5P₂ is defective in this strain.

To further investigate a specific role for Fig4p in PI3,5P₂ turnover, the inventor used a hyperactive Fab1p allele, fab1-6. This mutant produces slightly elevated levels of PI3,5P₂ under basal conditions and responds fully to hyperosmotic shock. When fab1-6 is the sole copy of Fab1p in Δfig4 cells PI3,5P₂ is elevated to 50 units instead of 100 units, consistent with a requirement for Fig4p in Fab1p activation. This partial elevation does not diminish by 30 minutes, consistent with Fig4p also functioning as a lipid phosphatase specific for PI3,5P₂. Thus, it appears Fig4p may have dual functions: dephosphorylation of Fab1p to induce PI3,5P₂ synthesis, and subsequently function as a lipid phosphatase to modify the newly synthesized PI3,5P₂.

Thus, each of these gene products discussed above provides a distinct, important regulatory function in PI(3,5)P₂-mediated signaling. Thus, these observations provide the basis for both assessing alterations in the ability of an individual to respond to insulin, and the ability to intervene in these signaling pathways to overcome deficits that lead to diabetes.

I. PI(3,5)P₂ Signaling

All cells constantly react to their outside environment. Micro-organisms have complex systems both to enable nutrient intake, and to provide defenses against sudden changes in the environment. Cells in multicellular organisms also have complex systems to take up nutrients, to coordinate with other cells by secreting effectors such as hormones and cytokines, and to respond to specific effectors produced by other cell types. In both lower and higher eukaryotes, endocytosis and exocytosis play major roles in these types of cellular functions.

Endocytosis and exocytosis rely on a complex array of organelles. Anterograde transport of proteins and lipids is required for secretion and organelle biogenesis. While retrograde traffic restores proteins and lipids to their resident intracellular compartment. Thus, membrane transport must be rapid, yet accurate or self correcting. Notably, phosphatidylinositol polyphosphates (PPIs) have recently emerged as key regulators of many of these membrane trafficking pathways (Huijbregts et al., 2000). PPIs transiently appear at specific membranes at specific times. However, the roles that these molecules play in membrane traffic and other events is not well understood. Therefore, there is a need to develop further molecular insights into how PPI synthesis and turnover is regulated, and how these PPIs function in down stream events.

A. General Properties of PPIs

An important feature of PPIs is the number of sterically distinct compounds that can be generated via phosphorylation of inositol at any or all of its six hydroxyls. Also, specific kinases and phosphatases allow rapid interconversion between phosphorylated species. Likewise, lipases can rapidly convert selected PPIs to diacylglycerol and inositol phosphates, which are also signaling molecules.

PPIs play a regulatory role through the spatial and temporal control of their synthesis and turnover, and through specific binding proteins. For example, GFP probes for PI3P have revealed the transient appearance of PI3P on phagosomes (Vieira et al., 2001; Pattni et al., 2001). From the properties of PPIs, one can propose likely functions that they serve in membrane traffic. For instance, the rapidity with which they are synthesized means that they can bring proteins to a discrete domain at a specific time. Moreover, they can bring in several types of proteins that all recognize one phosphoinositide species. This would ensure that proteins function together only when a particular lipid is present. Several lines of evidence suggest that this may happen in endocytosis.

Furthermore, having distinct, yet interconvertible phosphoinositides may achieve processivity for membrane mediated events by having a signalling phosphoinositide also serve as a precursor to a second signaling phosphoinositide. For example, PI3P may first initiate downstream events and then serve as the precursor for PI3,5P2, which in turn effects further related, downstream processes. Finally the large, charged headgroup found on PPIs may contribute to membrane shape changes that occur during formation of membrane tubules, vesicle formation and fission.

B. The Roles of PPIs in Membrane Traffic

At least four PPIs play a direct role in membrane traffic (Simonsen et al., 2001; Cullen et al., 2001). PI3P is required for protein sorting from the Golgi to the lysosome and is also required for autophagy. PI4P is required for formation of Golgi derived secretory vesicles. PI4,5P2 is required for exocytosis and endocytosis. PI3,5P2 is required for retrograde traffic from the vacuole to the late endosome. These lipids function through the action of PPI-binding proteins. The lipids themselves, through their bulky, charged head groups, may also contribute to membrane curvature. Several phosphatidylinositide binding domains have been discovered, and the recent pace of their discovery suggests that there are many more phosphatidylinositide-binding modules to be identified. Known binding domains include: FYVE domain (specific for PI3P), PX domain (some specific for PI3P, others for P13,4P2), ENTH domain (specific for P14,5P2), PH domain (some specific for P14,5P2, others for P13,4,5P3). A domain that specifically recognizes PI3,5P2 has not yet been found.

In addition to their roles in membrane traffic, PPIs play key roles in the regulation of several other cellular processes including: cell-growth and differentiation (Joly et al., 1995; Toker and Cantley, 1997; Cantley and Neel, 1999); Ca⁺⁺ mobilization10; regulation of selected ion channels (Huang et al., 1998; Sui et al., 1998; Hardie et al., 2001); cytoskeletal rearrangements (Lanier and Gertler, 2000). Until recently, it was thought that a single phosphoinositide may play multiple, unrelated regulatory roles. However, in at least some cases, a single phosphoinositide may coordinate diverse molecular events that all contribute to the same process. An example is P14,5P2, which was initially thought to function separately to regulate endocytosis via regulation of clathrin coat assembly and to regulate actin polymerization. But recent studies from several laboratories suggest that it is through the coordination of clathrin coat formation combined with actin polymerization that endocytosis is achieved (reviewed in (Cremona and De Camilli, 2001; Schafer, 2002). Since P14,5P2 is required for both these processes, it appears that PI4,5P2 coordinates multiple complex events that result in endocytosis and that removal of PI4,5P2 terminates endocytosis. While there will be similarities to how PPIs function, the behavior of each PPI will have unique aspects. Moreover, there are distinct advantages to studying a given PPI. For example, study of PI4,5P2 is complicated by the fact that it acts both in endocytosis and exocytosis. In contrast PI3,5P2 is required for retrograde traffic from the vacuole, but not for anterograde traffic.

C. PI3,5P2 Function and Regulation

Yeast mutants unable to make PI3,5P2 are defective in vacuole inheritance. Analysis of these mutants revealed that PI3,5P2 functions in the following diverse processes; vacuole membrane fission, retrograde traffic from the vacuole and vacuole acidification. The defect in vacuole inheritance is caused by defects in vacuole membrane deformation. Tubulation of the vacuole membrane to form the segregation structure may be related mechanistically to vacuole membrane fission, and retrograde vesicle formation and fission. Membrane deformation can be readily observed in vacuole inheritance, where a lobe of the vacuole rapidly changes from a sphere to an elongated tubule.

PI3,5P2 is synthesized from PI3P by the PI3P 5-kinase, Fab1p (Gary et al., 1998). The inventor recently discovered that the levels of this lipid rapidly increase and then decrease in response to hyperosmotic stress. They now have determined that regulation of Fab1p plays a key role in determining the steady-state levels of PI3,5P2. Overexpression of Fab1p alone has no effect on PI3,5P2 levels suggesting that the activation of Fab1p requires other proteins (Gary et al., 2002). In fact, the inventor have recently identified three Fab1p regulators—Vac7p20, Vac14p18 and Fig4p.

Currently less is known about PI3,5P2 than the other phosphoinositides, yet it plays an equally critical role. PI3,5P2 functions late in the endocytic system and regulates many processes that are performed by the late endosome and lysosome. Moreover, fab1 mutants are barely viable, strongly suggesting that PI3,5P2 is important for normal cellular functions. The study of PI3,5P2 in yeast provides several technical advantages. (1) One can transiently induce a greater than 20-fold rise in PI3,5P2 levels with a physiological stimulus. This dramatic increase and rapid decrease provides us with clear-cut methods to elucidate the regulation of PI3,5P2 synthesis and turnover. Furthermore, this extremely large increase may allow us to detect changes in localization of this lipid that might not be detectable in other systems. Moreover the osmotic stress stimulus appears to have a physiological basis and thus will aid in our ability to study how PI3,5P2 regulates downstream events. (2) The inventor has found that vacuole morphology strictly correlates with PI3,5P2 levels, providing a simple in vivo assay to monitor how growth conditions or mutated proteins effect PI3,5P2 levels. (3) The inventor's studies strongly suggest that in yeast PI3,5P2 synthesis and function is confined to the late endosome and vacuole. Thus, all the changes detected in total cells reflect changes on this confined set of membranes.

In addition to its presence in yeast, PI3,5P2 is present in all mammalian cell types tested (Whiteford et al., 1997; Dove et al., 1997; Jones et al., 1999). A mouse orthologue of Fab1p, PIKfyve (mFab1), has been identified and partially characterized (Sbrissa et al., 1999). The sequence similarity to yeast Fab1p is high and it is a true orthologue because (i) mFab1 partially restores PI3,5P2 levels in a yeast fab1Δ mutant (McEwen et al., 1999); (ii) isolated mFab1 produces PI3,5P2 from PI3P in vitro (Sbrissa et al., 1999); and (iii) transfection of fibroblasts with dominant-negative mFab1 causes vacuolation, which is reminiscent of the large vacuole phenotype observed in fab1 Ikonomov et al., 2001). Thus, PI3,5P2 likely plays fundamental roles in mammalian cells that are similar to its roles in yeast, i.e., ion homeostasis and membrane trafficking from the late endosome or lysosome. In addition, recent studies hint that mammlian Fab1 might be important in GLUT4 translocation: (1) mFab1 mRNA levels are elevated in insulin target tissues and become elevated during 3T3-L1 adipocyte differentiation (Shisheva et al., 1999); (2) mFab1 localization changes in response to insulin (Shisheva et al., 2001); (3) hyperosmotic stress (which in yeast causes a 20-fold elevation in PI3,5P2 levels) causes GLUT4 translocation to the plasma membrane in both adipocyte and muscle cell-lines (Chen et al., 1997; Janez et al., 2000a; Janez et al., 2000b; Li et al., 2001).

II. Insulin-Sensing Cells

Adipocytes are fat cells which play a central role in the development of many metabolic diseases including diabetes, obesity, and both familial and acquired lipodystrophy. Previous studies have demonstrated important roles for hormones, receptors and/or transcription factors, such as peroxisome proliferator-activated receptor gamma (PPAR-gamma) and CCAAT box enhancer binding protein alpha (C/EBP-alpha), in fat cell differentiation. The stimulation of adipose tissue glucose metabolism, which is ultimately responsible for bringing about postabsorptive blood glucose clearance, is the primary clinically relevant action of insulin. Insulin acts on many steps of glucose metabolism, but one of the most important effects is its ability to increase the rate of cellular glucose transport. This results from the translocation of the insulin-responsive transporter isoform, GLUT4, from intra-cellular vesicular storage sites to the plasma membrane. In adipocytes, a substantial amount of cellular GLUT4 is located in a specific highly insulinresponsive storage pool, termed GLUT4 Storage Vesicles (GSVs). GLUT4 can also translocate to the plasma membrane from the recycling endosomal pool which also additionally contains the GLUT1 isoform of glucose transporter and the transferrin receptor.

Muscle cells, though in many ways quite distinct from adipocytes, also exhibit the ability to respond to insulin. However, the purpose of this response is quite different from the storage role seen with adipocytes. In muscle cells, insulin facilitates the transport of glucose from the blood into the muscle where it can be manufactured into glycogen. The manufacture of muscle glycogen from glucose requires the presence of an enzyme called glycogen synthetase. Insulin also stimulates this enzyme, enabling the glycogen manufacturing process to proceed at a faster rate. Assuming that sufficient amounts of carbohydrate are available, the greater the plasma insulin levels after exercise, the faster the transport of glucose into the muscle cell and the faster the rate of muscle glycogen synthesis.

Given the key role of these cells in the insulin response, they are of particular interest in the present invention. For example, in assessing the ability of a drug to alter the insulin response, adipocytes and muscle cells will provide an obvious choice in constructing a meaningful assay. Similarly, by examining the behavior of muscle cells and adipocytes when stimulated by insulin, one can assess an individual suceptibility to diabetes. In addition, when attempting to alter insulin response, targeting of therapies to these cells will likely be important.

III. Fab1p, Vac14p and Fig4p

The Saccharomyces cerevisiae FAB1 gene encodes the sole phosphatidylinositol 3-phosphate [PtdIns(3)P] 5-kinase responsible for synthesis of the polyphosphoinositide PtdIns(3,5)P₂. fab1 null mutants have dramatically enlarged vacuoles and cannot grow at elevated temperatures. Additionally, mutants in vac7, a 128-kDa transmembrane protein that localizes to vacuolar membranes, have nearly undetectable levels of PtdIns(3,5)P2, suggesting that Vac7 functions to regulate Fab1 kinase activity. Two other proteins—Vac14 and Fig4—are the activators of Fab1. Fig4 also has a second role as a lipid phosphatase that turns over PI3,5P2.

A fab1 mutant allele that bypasses the requirement for Vac7 in PtdIns(3,5)P2 production has been created. Expression of this fab1 allele in vac7Δ mutant cells suppresses the temperature sensitivity, vacuolar morphology, and PtdIns(3,5)P₂ defects normally exhibited by vac7Δ mutants. A mutant allele of FIG4, whose gene product contains a Sac1 polyphosphoinositide phosphatase domain, has been identified which suppresses vac7Δ mutant phenotypes. Specific mutations of the FIG4 gene in vac7Δ mutant cells suppresses the temperature sensitivity and vacuolar morphology defects, and dramatically restores PtdIns(3,5)P₂ levels. These results suggest that generation of PtdIns(3,5)P₂ by the Fab1 lipid kinase is regulated by Vac7, whereas turnover of PtdIns(3,5)P₂ is mediated in part by the Sac1 polyphosphoinositide phosphatase family member Fig4.

The Saccharomyces cerevisiae Class III vac mutant vac14-1, was isolated via fluorescence-activated cell sorting (for description of approach see Wang et al., 1996). Like vac7 and fab1 mutants, vac14-1 cells are defective in vacuole inheritance, acidification, and morphology. These mutants have a single, unlobed, enlarged vacuole. Frequently, the vacuole spans both the mother and daughter cell resulting in an “open figure eight” vacuole morphology (Bonangelino et al., 1997).

The VAC14 open reading frame is YLR386W (sequence deposited by the Yeast Genome Sequencing Project). The VAC14 2.64-kb open reading frame encodes a polypeptide of 880 amino acids. There are no notable motifs except for a putative transmembrane domain. However, VAC14 displays a high degree of identity with open reading frames present in other eukaryotic organisms. The two regions of highest identity are near the NH2 terminus (residues 1-171) and COOH terminus (residues 578-746).

Both mouse and human sequences matching either end were identified in the corresponding EST databases. Moreover, a human hypothetical protein, FLJ10305, found on chromosome 16, shows a high degree of identity with the COOH-terminal region of Vac14p and its sequence matches human EST sequences. Both the NH2— and COOH-terminal ESTs map to human chromosome 16, suggesting that they correspond to the same gene. No obvious Vac14p homologues have been found in any of the published bacterial genomes.

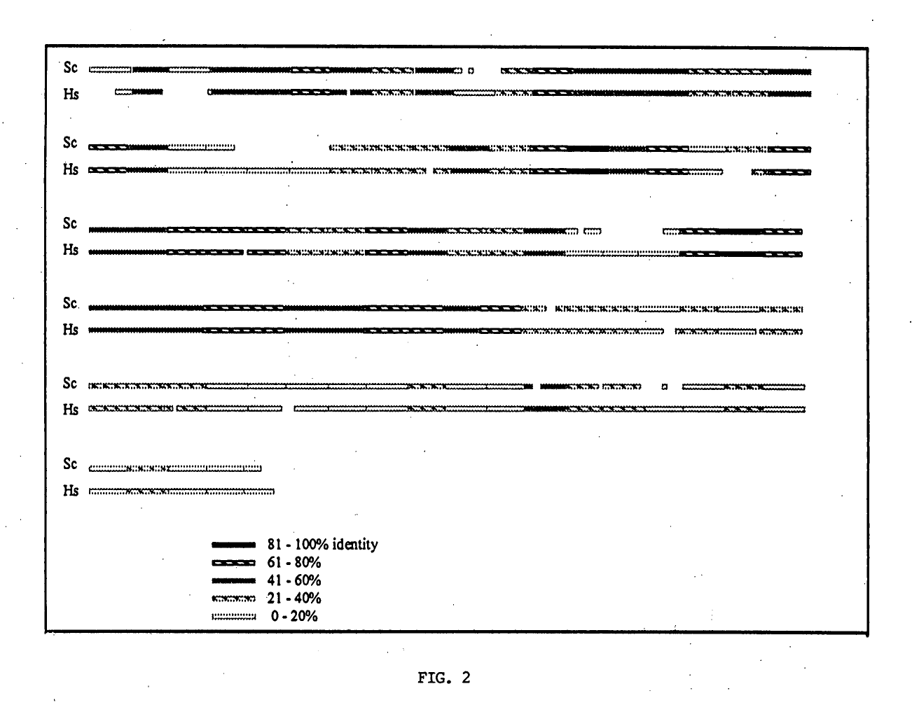

Fig4 is one of four proteins in yeast that contains a polyphosphoinositide phosphatase domain called the Sac1 domain (Guo et al., 1999) (FIG. 2). Other representatives include Sac1 and the inositide polyphosphate 5-phosphatases Sjl2/Inp52 and Sjl3/Inp53 (Srinivasan et al., 1997; Stolz et al., 1998a,b; Guo et al., 1999). In vitro, the Sac1 domains from Sac1 and Sjl3 are able to dephosphorylate PtdIns(3,5)P2, PtdIns(3)P, and PtdIns(4)P, but do not appear to recognize PtdIns(4,5)P2 (Guo et al., 1999; Hughes et al., 2000). Furthermore, sac1 mutants accumulate approximately 2.5-fold higher levels of PtdIns(3,5)P2 than wild-type cells (Guo et al., 1999).

As discussed above, a sac1Δ vac7Δ double mutant strain was able to grow at 38° C., whereas sjl2Δ vac7Δ and sil3Δ vac7Δ double mutant strains were not able to grow. There is also unpublished data indicating that in a fig4-knockout strain, Fab1 is not properly dephosphorylated in response to osmotic stress. As expected, neither deletion of SJL2 nor SJL3 was able to rescue PtdIns(3,5)P2 levels in vac7Δ mutant cells. In contrast, the PtdIns(3,5)P2 levels in the vac7Δ sac1Δ double mutant strain were higher than in vac7Δ fig4Δ double-mutant cells. However, in addition to the elevation in the level of PtdIns(3,5)P2, the level of PtdIns(4)P also was dramatically elevated, approximately 16-fold more than in wild-type or vac7Δ cells. Furthermore, transformation of the SAC1 gene in the remaining bvs mutants did not reverse the vac7Δ suppression, suggesting that mutations in SAC1 were not isolated. Sac1 is an integral membrane protein localized to the endoplasmic reticulum in yeast that primarily dephosphorylates PtdIns(4)P in vivo (Guo et al., 1999; Foti et al., 2001). These results indicate that inhibition of PtdIns(3,5)P2 turnover by inactivation or mutation of Fig4 or Sac1 can restore PtdIns(3,5)P2 levels to allow the bypass of Vac7 function. A further observation involves the use of a partially activated Fab1 mutant, fab1-6. In fig4Δ/fab1-6 cells, the PtdIns(3,5)P2 defect is less dramatic, and it is easier to monitor the turnover of PtdIns(3,5)P2, which clearly is defective.

In sum, Fig4 appears to have lipid phosphatase activity, is considered to be an activator of Fab1 (although it remains unclear whether this is a direct effect), and plays a role in PtdIns(3,5)P2 turnover.

IV. Peptides and Polypeptides

Thus, in one aspect, the present invention relates to proteinaceous materials for Fab1p, Vac14p, and Fig4. In addition to an entire polypeptide (SEQ ID NOS:1, 3, 5 and 7), the present invention also relates to fragments of the polypeptides that may or may not retain various of the functions described below. Fragments, including the N-terminus of the molecule may be generated by genetic engineering of translation stop sites within the coding region (discussed below). Alternatively, treatment of the STARS with proteolytic enzymes, known as proteases, can produce a variety of N-terminal, C-terminal and internal fragments. Examples of fragments may include contiguous residues of SEQ ID NOS:1, 3, 5, and 7 of 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, 200, 300, 400 or more amino acids in length. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Variants

Amino acid sequence variants of the above-noted polypeptides can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventor that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *-0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within +1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson et al., 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of the native polypeptides, but with altered and even improved characteristics.

B. Domain Switching

Domain switching involves the generation of chimeric molecules using different but, in this case, related polypeptides. These molecules may have additional value in that these “chimeras” can be distinguished from natural molecules, while possibly providing the same function.

C. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

D. Purification of Proteins

It will be desirable to purify polypeptides or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Synthetic Peptides

The present invention also describes peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

F. Antigen Compositions

The present invention also provides for the use of proteins or peptides as antigens for the immunization of animals relating to the production of antibodies. It is envisioned that Fab1p, Vac14p, Fig4p, or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

V. Nucleic Acids

The present invention also provides, in another embodiment, nucleic acids encoding Fab1p, Vac14p and Fig4p. Genes from human and yeast have been identified, and in some cases mouse as well. See, for example, SEQ ID NOS: 2, 4, 6, and 8 respectively. The present invention is not limited in scope to these genes, however, as one of ordinary skill in the could, using these nucleic acids, readily identify related homologs in these and various other species (e.g., rat, rabbit, dog, monkey, gibbon, chimp, ape, baboon, cow, pig, horse, sheep, cat and other species).

In addition, it should be clear that the present invention is not limited to the specific nucleic acids disclosed herein. As discussed below, a nucleic acid may contain a variety of different bases and yet still produce a corresponding polypeptide that is functionally indistinguishable, and in some cases structurally, from the human and yeast genes disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance activity.

A. Polynucleotides

Nucleic acids according to the present invention may encode an entire Fab1p, Vac14p, Fig4p gene, a domain of Fab1p, Vac14p, Fig4p, or any other fragment of Fab1p, Vac14p, Fig4p as set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given Fab1p, Vac14p, or Fig4p from a given species may be represented by natural variants that have slightly different nucleic acid sequences but, nonetheless, encode the same protein (see Table 1 below).

As used in this application, the term “a nucleic acid encoding a Fab1p, Vac14p, Fig4p” refers to a nucleic acid molecule that has been isolated free of total cellular nucleic acid. In preferred embodiments, the invention concerns a nucleic acid sequence essentially as set forth in SEQ ID NOS: 2, 4, 6, or 8 (mouse, human, zebrafish, or C. elegans, respectively). The term “as set forth in SEQ ID NOS: 2, 4, 6, or 8” means that the nucleic acid sequence substantially corresponds to a portion of SEQ ID NO:2, 4, 6, or 8. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NOS:2, 4, 6, or 8 are contemplated. Sequences that are essentially the same as those set forth in SEQ ID NOS:2, 4, 6, or 8 may also be functionally defined as sequences that are capable of hybridizing to a nucleic acid segment containing the complement of SEQ ID NOS:2, 4, 6 or 8 under medium or high stringency conditions.

The DNA segments of the present invention include those encoding biologically functional equivalent STARS proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Oligonucleotide Probes and Primers

Naturally, the present invention also encompasses DNA segments that are complementary, or essentially complementary, to the sequence set forth in SEQ ID NOS:2, 4, 6, or 8. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NOS:2, 4, 6, or 8 under relatively stringent conditions such as those described herein. Such sequences may encode entire proteins or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 5000 bases and longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to Fab1p, Vac14p, or Fig4p, or, more particularly, homologs from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single-stranded and double-stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

E. RNAi

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides +3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et. al. wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen, et. al., 2000; Elbashir et. al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

F. Vectors for Cloning, Gene Transfer and Expression

Within certain embodiments expression vectors are employed to express a STARS polypeptide product, which can then be purified and, for example, be used to vaccinate animals to generate antisera or monoclonal antibody with which further studies may be conducted. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

(i) Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct. Additionally, any other promoter/enhancer combination (for example, as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

(ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(iii) Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornaovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

(iv) Delivery of Expression Constructs

There are a number of ways in which expression constructs may be introduced into cells. In certain embodiments of the invention, a vector (also referred to herein as a gene delivery vector) is employed to deliver the expression construct. By way of illustration, in some embodiments, the vector comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene delivery vectors were DNA viruses including the papovaviruses (simian virus 40, bovine papilloma virus, and polyoma) (Ridgeway, 1988; Baichwal and Sugden, 1986). Generally, these have a relatively low capacity for foreign DNA sequences and have a restricted host spectrum. They can accommodate only up to 8 kb of foreign genetic material but can be readily introduced in a variety of cell lines and laboratory animals (Nicolas and Rubenstein, 1988; Temin, 1986). Where viral vectors are employed to deliver the gene or genes of interest, it is generally preferred that they be replication-defective, for example as known to those of skill in the art and as described further herein below.

The expression vector may comprise a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage and are able to infect non-dividing cells. So far, adenoviral infection appears to be linked only to mild disease such as acute respiratory disease in humans.

Adenovirus is particularly suitable for use as a gene delivery vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is important to minimize this possibility by, for example, reducing or eliminating adnoviral sequence overlaps within the system and/or to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of such adenovirus vectors is about 7.5 kb, or about 15% of the total length of the vector. Additionally, modified adenoviral vectors are now available which have an even greater capacity to carry foreign DNA.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, a preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be selected from any of the 42 different known serotypes or subgroups A-F. Adenovirus type 5 of subgroup C is a preferred starting material for obtaining a replication-defective adenovirus vector for use in the present invention. This is, in part, because Adenovirus type 5 is a human adenovirus about which a great deal of biochemical and genetic information is known, and it has historically been used for most constructions employing adenovirus as a vector.

As stated above, a preferred adenoviral vector according to the present invention lacks an adenovirus E1 region and thus, is replication. Typically, it is most convenient to introduce the polynucleotide encoding the gene of interest at the position from which the E1-coding sequences have been removed. However, the position of insertion of the construct within the adenovirus sequences is not critical to the invention. Further, other adenoviral sequences may be deleted and/or inactivated in addition to or in lieu of the E1 region. For example, the E2 and E4 regions are both necessary for adenoviral replication and thus may be modified to render an adenovirus vector replication-defective, in which case a helper cell line or helper virus complex may employed to provide such deleted/inactivated genes in trans. The polynucleotide encoding the gene of interest may alternatively be inserted in lieu of a deleted E3 region such as in E3 replacement vectors as described by Karlsson et al. (1986), or in a deleted E4 region where a helper cell line or helper virus complements the E4 defect. Other modifications are known to those of skill in the art and are likewise contemplated herein.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies indicated that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include administration via intracoronary catheter into one or more coronary arteries of the heart (Hammond, et al., U.S. Pat. Nos. 5,792,453 and 6,100,242) trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact—sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This indicated that large portions of the genome could be replaced with foreign genetic material. The hepatotropism and persistence (integration) were particularly attractive properties for liver-directed gene transfer. Chang et al., recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. In general, viral vectors accomplish delivery of the expression construct by infecting the target cells of interest. Alternatively to incorporating the expression construct into the genome of a viral vector, the expression construct may be encapsidated in the infectious viral particle.

Several non-viral gene delivery vectors for the transfer of expression constructs into mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression vector may simply consist of naked recombinant DNA or plasmids comprising the expression construct. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention, transferring of a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome, another non-viral gene delivery vector. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0 273 085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

VI. Transgenic Animals

A particular embodiment of the present invention provides transgenic animals that contain heterologous gentic construct. For example, one may attache a selectable or screenable marker protein to a Fab1p, Vac14p or Fig4p promoter. Alternatively, transgenic knockouts and animals expressing a Fab1p, Vac14p or Fig4p under the control of an inducible, or recombinant cell lines derived from such animals, may be useful in creating a disease model of diabetes. The use of animals or cells constitutively expressing Fab1p, Vac14p or Fig4p also may provide useful models.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), Brinster et al. (1985); which is incorporated herein by reference in its entirety) and in Hogan et al. (1994).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA. Other methods for purification of DNA for microinjection are described in Palmiter et al. (1982); and in Sambrook et al. (1989).

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

VII. Diagnostic Methods

The inventor believes that Fab1p, Vac14 and Fig4p play an important role in the ability of cells to respond to insulin. Thus, in another embodiment, there are provided methods for diagnosing defects in Fab1p, Vac14 and Fig4p expression and function. More specifically, point mutations, deletions, insertions or regulatory pertubations relating to Fab1p, Vac14 and Fig4p, as well as increases or decrease in levels of expression, may be assessed using standard technologies, as described below.

A. Genetic Diagnosis

One embodiment of the instant invention comprises a method for detecting variation in the expression of Fab1p, Vac14 and Fig4p. This may comprise determining the level of Fab1p, Vac14 and Fig4p or determining specific alterations in the expressed product.

A suitable biological sample can be any tissue or fluid that contains genetic material. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, sputum, cerebrospinal fluid, lacrimal fluid, stool or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects may be identified by the present methods. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of Fab1p, Vac14 or Fig4p produced, both by altering the transcription of the gene or in destabilizing or otherwise altering the processing of either the transcript (mRNA) or protein.

It is contemplated that other mutations in the Fab1p, Vac14 and Fig4p genes may be identified in accordance with the present invention. A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™-SSCP.

(i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ¹⁴C, ³⁵S, ³H, or other label), with a fluorophore (rhodamine, fluorescein) or a chemillumiscent (luciferase).

(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989), incorporated herein by reference in its entirety.

(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

(iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the STARS genes that may then be analyzed by direct sequencing.

(vi) Kit Components

All the essential materials and reagents required for detecting and sequencing STARS and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

B. Immunologic Diagnosis

Antibodies of the present invention can be used in characterizing the Fab1p, Vac14 and Fig4p content of healthy and diseased tissues, through techniques such as ELISAs and Western blotting. This may provide a screen for the presence or absence of type 2 diabetes.

The use of antibodies of the present invention, in an ELISA assay is contemplated. For example, antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for Fab1p, Vac14 or Fig4p that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° C. to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 h at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

VIII. Screening Methods

The present invention also contemplates the screening of compounds for various abilities to interact with and/or affect Fab1p, Vac14p and/or Fig4p expression or function. Particularly preferred compounds will be those useful in inhibiting or promoting the actions of Fab1p, Vac14p and/or Fig4p in regulating insulin response. In the screening assays of the present invention, the candidate substance may first be screened for basic biochemical activity—e.g., binding to a target molecule—and then tested for its ability to inhibit modulate activity, at the cellular, tissue or whole animal level.

A. Modulators and Assay Formats

i) Assay Formats

The present invention provides methods of screening for modulators of Fab1p, Vac14p or Fig4p expression and activity. In one embodiment, the present invention is directed to a method of:
(a) providing an active Fab1p, Vac14p and/or Fig4p preparation;
(b) contacting said preparation with a candidate modulator; and
(c) measuring a Fab1p, Vac14p and/or Fig4p related activity.

In yet another embodiment, the assay looks not at function, but at expression. Such methods would comprise, for example:
(a) providing a cell in which a Fab1p, Vac14p and/or Fig4p promoter directs the expression of a polypeptide;
(b) contacting said cell with a candidate modulator; and
(c) measuring the effect of said candidate modulator on expression of said polypeptide.

The polypeptide may be Fab1p, Vac14p and/or Fig4p, or it may be an indicator protein.

In another embodiment, the present invention is directed to a method of:
(a) providing a cell defective in a Fab1p, Vac14p and/or Fig4p function;
(b) contacting said preparation with a candidate modulator; and
(c) measuring the activation of a Fab1p, Vac14p and/or Fig4p related activity.

This sort of assay would provide a model for determining if a drug could overcome a Fab1p, Vac14p and/or Fig4p related block in insulin response.

ii) Candidate Substances

As used herein, the term “candidate substance” refers to any molecule that may potentially modulate Fab1p, Vac14p and/or Fig4p expression or function. The candidate substance may be a protein or fragment thereof, a small molecule inhibitor, or even a nucleic acid molecule. The term “candidate modulator” may be used in place of “candidate substance.” It may prove to be the case that the most useful pharmacological compounds will be compounds that are structurally related to compounds which interact naturally with Fab1p, Vac14p and/or Fig4p. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like Fab1p, Vac14p and/or Fig4p, and then design a molecule for its ability to interact with Fab1p, Vac14p and/or Fig4p. Alternatively, one could design a partially functional fragment of Fab1p, Vac14p and/or Fig4p (binding, but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of hypertrophic response. Other suitable inhibitors include antisense molecules, RNAi, ribozymes, and antibodies (including single chain antibodies).

It will, of course, be understood that the screening methods of the present invention are useful in themselves notwithstanding the fact that effective candidates may not be found. The invention provides methods for screening for such candidates, not solely methods of finding them.

B. In Vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a Fab1p, Vac14p and/or Fig4p protein or fragment thereof is provided.

The target may be either free in solution, fixed to a support, expressed in or on the surface of a cell. Either the target or the compound may be labeled, thereby permitting determining of binding. In another embodiment, the assay may measure the inhibition of binding of a target to a natural or artificial substrate or binding partner (such as Fab1p, Vac14p and/or Fig4p). Competitive binding assays can be performed in which one of the agents (Fab1p, Vac14p and/or Fig4p for example) is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, Fab1p, Vac14p or Fig4p, and then washed fron the support to remove non-specifically bound protein. Bound polypeptide is detected by various methods.

Purified target, such as Fab1p, Vac14p or Fig4p, can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link an active region to a solid phase.

C. In Cyto Assays

Various cell lines that express Fab1p, Vac14p and/or Fig4p can be utilized for screening of candidate substances. For example, cells containing Fab1p, Vac14p and/or Fig4p with an engineered indicators can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (growth, size, Ca⁺⁺ effects). Alternatively, molecular analysis may be performed in which the function of Fab1p, Vac14p and/or Fig4p and related pathways may be explored. This involves assays such as those for protein expression, enzyme function, substrate utilization, mRNA expression (including differential display of whole cell or polyA RNA) and others.

D. In Vivo Assays

The present invention particularly contemplates the use of various animal models. Transgenic animals may be created with constructs that permit Fab1p, Vac14p and/or Fig4p expression and activity to be controlled and monitored. The generation of these animals has been described elsewhere in this document.

Treatment of these animals with test compounds will involve the administration of the compound, in an appropriate form, to the animal. Administration will be by any route the could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

E. Production of Modulators

In an extension of any of the previously described screening assays, the present invention also provide for methods of producing modulators. The methods comprising any of the preceding screening steps followed by an additional step of “producing the candidate substance identified as a modulator of” the screened activity.

IX. Therapies

A. Methods of Treatment

In accordance with the present invention, it is further contemplated that use of a modulator to alter the expression of one or more of Fab1p, Vac14p and/or Fig4p, or to alter the activity of one or more of Fab1p, Vac14p and/or Fig4p, will prove useful in treating diabetes.

In a particular embodiment, where a defect in Fab1p, Vac14p and/or Fig4p is identified, one may employ a gene therapy approach where the corresponding gene is provided to the appropriate population of insulin-sensing cells. Delivery of expression constructs is discussed elsewhere in this document. Alternatively, it may be that in certain cases, the overexpression of Fab1p, Vac14p and/or Fig4p creates an improper response to insulin, in which case on may choose to downregulate one or more of these targets using the aforementioned techniques (antisense, ribozymes, RNAi, single chain antibodies, etc.).

In another therapeutic embodiment, the use of certain drugs to activate the insulin response pathway in cells such as adipocytes or muscle cells, despite the existence of defects in Fab1p, Vac14p and/or Fig4p, will find use. Such drugs may be identified through the use of screening assays as described in other portions of this document.

B. Pharmaceutically Acceptable Formulations

Where clinical applications are contemplated, it will be necessary to prepare pharmaceutical compositions of the cells in a form appropriate for transplant. The cells will generally be prepared as a composition that is essentially free of pyrogens, as well as other impurities that could be harmful to humans or animals.

One will generally desire to employ appropriate salts and buffers to render stable cells suitable for introduction into a patient. Aqueous compositions of the present invention comprise an effective amount of stable cells dispersed in a pharmaceutically acceptable carrier or aqueous medium, and preferably encapsulated.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. As used herein, this term is particularly intended to include biocompatible implantable devices and encapsulated cell populations. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

Under ordinary conditions of storage and use, the cell preparations may further contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

An effective amount of a therapeutic composition is determined based on the intended goal. The term “unit dose” refers to a physically discrete unit suitable for use in a subject, each unit containing a predetermined quantity of the cell composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

X. Adjunct Therapies and Related Procedures

In accordance with the present invention, it may prove advantageous to combine the methods disclosed herein with adjunct therapies or procedures to enhance the overall therapeutic effect. Such therapies and procedures are set forth in general, below. A skilled physician will be apprised of the most appropriate fashion in which these therapies and procedures may be employed.

A. Supplemental Insulin Therapy

The present invention, though designed to eliminate the need for other therapies, may work well in combination with traditional insulin supplementation. Such therapies should be tailored specifically for the individual patient given their current clinical situation, and particularly in light of the extent to which transplanted cells can provide insulin. The following are general guidelines for typical a “monotherapy” using insulin supplementation by injection.

Insulin can be injected in the thighs, abdomen, upper arms or gluteal region. In children, the thighs or the abdomen are preferred. These offer a large area for frequent site rotation and are easily accessible for self-injection. Insulin injected in the abdomen is absorbed rapidly while from the thigh it is absorbed more slowly. Hence, patients should not switch from one area to the other at random. The abdomen should be used for the time of the day when a short interval between injection and meal is desired (usually prebreakfast when the child may be in a hurry to go to school) and the thigh when the patient can wait 30 minutes after injection for his meal (usually predinner). Within the selected area systematic site rotation must be practiced so that not more than one or two injections a month are given at any single spot. If site rotation is not practiced, fatty lumps known as lipohypertrophy may develop at frequently injected sites. These lumps are cosmetically unacceptable and, what is more important, insulin absorption from these regions is highly erratic.

Before injecting insulin, the selected site should be cleaned with alcohol. Injecting before the spirit evaporates can prove to be quite painful. The syringe is held like a pen in one hand, pinching up the skin between the thumb and index finger of the other hand, and inserting the needle through the skin at an angle of 45-90° to the surface. The piston is pushed down to inject insulin into the subcutaneous space (the space between the skin and muscle), then one waits for a few seconds after which release the pinched up skin before withdrawing the needle. The injection site should not be massaged.

For day-to-day management of diabetes, a combination of short acting and intermediate acting insulin is used. Some children in the first year after onset of diabetes may remain well controlled on a single injection of insulin each day. However, most diabetic children will require 2,3 or even 4 shots of insulin a day for good control. A doctor should decide which regimen is best suited.

One injection regimen: A single injection comprising a mix of short acting and intermediate acting insulin (mixed in the same syringe) in 1:3 or 1:4 proportion is taken 20 to 30 minutes before breakfast. The usual total starting dose is 0.5 to 1.0 units/kg body weight per day. This regimen has three disadvantages: (1) all meals must be consumed at fixed times; (2) since the entire quantity of insulin is given at one time, a single large peak of insulin action is seen during the late and early evening hours making one prone to hyopglycemia at this time; (3) as the action of intermediate acting insulin rarely lasts beyond 16-18 hours, the patient's body remains underinsulinized during the early morning hours, the period during which insulin requirement in the body is actually the highest.

Two-injection regimen: This regimen is fairly popular. Two shots of insulin are taken—one before breakfast (⅔ of the total dose) and the other before dinner (⅓ of the total dose). Each is a combination of short acting and intermediate acting insulin in the ratio of 1:2 or 1:3 for the morning dose, and 1:2 or 1:1 for the evening dose. With this regimen the disadvantages of the single injection regimen are partly rectified. Some flexibility is possible for the evening meal. Further, as the total days' insulin is split, single large peaks of insulin action do not occur hence risk of hypoglycemia is reduced and one remains more or less evenly insulinized throughout the day. On this regimen, if the pre-breakfast blood glucose is high, while the 3 a.m. level is low, then the evening dose may need to be split so as to provide short acting insulin before dinner and intermediate acting insulin at bedtime.

Multi-dose insulin regimens: The body normally produces insulin in a basal-bolus manner, i.e., there is a constant basal secretion unrelated to meal intake and superimposed on this there is bolus insulin release in response to each meal. Multi-dose insulin regimens were devised to mimic this physiological pattern of insulin production. Short acting insulin is taken before each major meal (breakfast, lunch and dinner) to provide “bolus insulin” and intermediate acting insulin is administered once or twice a day for “basal insulin.” Usually bolus insulin comprises 60% of the total dose and basal insulin makes up the remaining 40%. With this regimen you have a lot of flexibility. Both the timing as well as the quantity of each meal can be altered as desired by making appropriate alterations in the bolus insulin doses. To take maximum advantage of this regimen, one should learn “carbohydrate counting” and work out carbohydrate:insulin ratio—the number of grams of carbohydrate for which the body needs 1 unit of insulin.

B. Monitoring Glucose Levels

Any person suffering from diabetes will be very familiar with the need to regularly measure blood glucose levels. Blood glucose level is the amount of glucose, or sugar, in the blood. It is also is referred to as “serum glucose level.” Normally, blood glucose levels stay within fairly narrow limits throughout the day (4 to 8 mmol/l), but are often higher after meals and usually lowest in the morning. Unfortuantely, when a person has diabetes, their blood glucose level sometimes moves outside these limits. Thus, much of a diabetic's challenge is to When one suffers from diabetes, it is important that glucose level be as near normal as possible. Stable blood glucose significantly reduces the risk of developing late-stage diabetic complications, which start to appear 10 to 15 years after diagnosis with Type 1 diabetes, and often less than 10 years after diagnosis with Type 2 diabetes.

Blood glucose levels can be measured very simply and quickly with a home blood glucose level testing kit, consisting of a measuring device itself and a test strip. To check blood glucose level, a small amount of blood is placed on the test strip, which is then placed into the device. After about 30 seconds, the device displays the blood glucose level. The best way to take a blood sample is by pricking the finger with a lancet. Ideal values are (a) 4 to 7 mmol/l before meals, (b) less than 10 mmol/l one-and-a-half hours after meals; and (c) around 8 mmol/l at bedtime.

People who have Type 1 diabetes should measure their blood glucose level once a day, either in the morning before breakfast or at bedtime. In addition, a 24-hour profile should be performed a couple of times a week (measuring blood glucose levels before each meal and before bed). People who have Type 2 diabetes and are being treated with insulin should also follow the schedule above. People who have Type 2 diabetes and who are being treated with tablets or a special diet should measure their blood glucose levels once or twice a week, either before meals or one-and-a-half hours after a meal. They should also perform a 24-hour profile once or twice a month.

The main advantage for measuring blood glucose levels of insulin-treated diabetics in the morning is that adjusted amounts of insulin can be taken if the blood glucose level is high or low, thereby reducing the risk of developing late-stage diabetic complications. Similarly, the blood glucose level at bedtime should be between 7 and 10 mmol/l. If blood glucose is very low or very high at bedtime, there may be a need to adjust food intake or insulin dose. Blood glucose should also be measured any time the patient does not feel well, or think blood glucose is either too high or too low. People who have Type 1 diabetes with a high level of glucose in their blood (more than 20 mmol/l), in addition to sugar traces in the urine, should check for ketone bodies in their urine, using a urine strip. If ketone bodies are present, it is a warning signal that they either have, or may develop, diabetic acidosis.

### XI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials And Methods

Media, strains, and molecular biology techniques. Yeast extract-peptone-dextrose (YEPD) media, synthetic minimal media with the necessary nutritional auxotrophic supplements, sporulation media, synthetic complete media without inositol, and synthetic media without uracil or inositol (Sherman, 1991; Schu et al., 1993) were prepared as described. High pH YEPD plates with 1.8 M ethylene glycol contained: 1% yeast extract, 2% Bacto-peptone, 1.5% agar, 2% glucose, 1.8 M ethylene glycol, and 100 mM potassium phosphate (pH 7.6). The last three ingredients were not autoclaved and added separately.

Labeling yeast vacuoles with FM4-64 or quinacrine. Yeast vacuoles were visualized in vivo by labeling log-phase cells with 80 μM N-(3-triethylammoniumpropyl)-4-(p-diethyl-aminophenylhexatrienyl) (FM4-64) (Bonangelino et al., 1997; Vida and Emr, 1995) or with quinacrine (Weisman et al., 1987). Cells were viewed with a 100× objective lens on an Olympus BX-60 fluorescence microscope (FM4-64: excitation, 560 nm, dichroic mirror at 595 nm, emission, 630 nm; quinacrine; excitation, 470 nm; dichroic mirror at 495 nm; emission, 425 nm) combined with a low level of transmitted light to reveal cell outlines. Images were captured digitally with a Hamamatsu ORCA CCD camera controlled with IP spectrum software (Scanalytics). Images were processed using Adobe Photoshop™.

Cloning VAC14. VA C14 was cloned by complementing an inability of vac14-1 cells to grow in high pH media with ethylene glycol at 33° C. (Bonangelino et al., 1997). 12 overlapping clones that complemented the growth and vacuolar morphology defects of vac14-1 cells were obtained. A 3.9-kb SpeI fragment containing a single complementing open reading frame was identified and partially sequenced. Confirmation that YLR386W (sequence deposited by the Yeast Genome Sequencing Project) is VAC14 was demonstrated by creating a strain with LEU2 linked to the wild-type YLR386W locus (see below).

Construction of a strain with LEU2 integrated adjacent to the VAC14 locus and a strain with a chromosomal deletion of VAC14. To construct a LEU2-marked strain, a 3.9-kb SpeI fragment containing VAC14 was subcloned into pRS305 (Sikorski and Hieter, 1989) (pCB48). This plasmid was linearized with XhoI and transformed into wild-type (LWY7235). PCR with genomic DNA isolated from the transformants (Davis et al., 1980) confirmed that integration occurred at the correct locus. The primers used were 5′-CGCGGCAGTATTGAGGG-3′ (v14A) and the T7 universal primer. Diploid cells were generated by crossing the LEU2-marked strain (VAC14) with LWY4552 (leu2,3-112, vac14-1). Of 16 tetrads analyzed, all LEU2⁺ colonies were wild-type, whereas all leu2⁻ cells displayed vac14-1 phenotypes.

To create a chromosomal deletion of VAC14, the 3.9-kb SpeI fragment, containing VAC14 and both 5′ and 3′ untranslated regions, was subcloned into pRS424 (pCB49). VAC14 was removed with StyI and BclI and replaced by the PCR-amplified TRP1 gene (digested with StyI and BamHI). The primers used to amplify TRP1 were V41 (5′-GCTACCCCTTGGGTC ACCTTACGTACAATCTTG-3′) and V42 (5′-CGGGATCCCACTCAACCCTATCTCGGTC-3′). The resulting plasmid (pCB52) was digested with SpeI and the 2.5-kb fragment (containing the VA C14 knockout cassette) was transformed into LWY3143. Colony PCR (with the primers v14A; see above) and V43 5′-GACTTGAAATTTTCCTTGC-3′) identified transformants containing TRP1 at the VAC14 locus.

Preparation of antibodies to Vac14p. A GST-VAC14 chimera was created by subcloning VAC14 into the IPTG inducible GST vector pET21a (Marshall et al., 1996). To introduce the appropriate restriction sites (XmaI at the 5′ end and XhoI at the 3′ end), VAC14 was amplified using the primers 5′-CGCGCCCCGGGGACCATGGAAAAATCGATTGCC-3′ and 5′-GGGCTCGAGGGGTTATTTTTTTAATTTATCGG-3′. The PCR product was digested and ligated to pET21a and digested with XmaI and SalI. The resulting plasmid (pCB69) was transformed into Escherichia coli DE3 cells. The soluble GST-Vac14 fusion protein was isolated on glutathione-Sepharose beads and antibodies were raised in goats (Elmira Biological). Serum was depleted of GST antibodies by passaging over a GST-Affi gel column (Bio-Rad Laboratories) and further purified by passage over a total yeast protein column prepared from vac14Δ cells.

Subcellular fractionation and Western blot Analysis. Yeast cell extracts and subcellular fractionations were performed as described (Bonangelino et al., 1997). Briefly, yeast strains were grown in YEPD media at 24° C. to an OD600 of 0.6. Cells were harvested, washed with cytosol buffer (20 mM Hepes, pH 6.8, 0.15 M potassium acetate, 10 mM MgCl2, and 0.25 M sorbitol), resuspended to approximately 100 OD/ml and lysed with glass beads. Cell extracts were centrifuged at 500 g at 4° C. for 4 min The S5 supernatants were centrifuged at 13,000 g at 4° C. for 10 min and the resulting S13 supernatants were centrifuged at 100,000 g at 4° C. for 1 h. The pellets (P13 and P100) were resuspended in an equal amount of cytosol cocktail as the supernatants. Laemmli sample loading buffer was added to equivalent OD units of each fraction. Samples were heated to 80° C. for 10 min before loading onto a 7.5% SDS-polyacrylamide gel. The proteins were transferred to nitrocellulose (ECL High Bond; Amersham Life Science, Piscataway, N.J.) in Tris-glycine-methanol transfer buffer at 4° C. at 34 V for 20 h. Detection of Vac14p was performed with goat anti-Vac14 antibodies at a 1:5,000 dilution. Donkey anti-goat IgG-horseradish peroxidase (Jackson ImmunoResearch Laboratories) at 1:5,000 dilution was the secondary antibody. Detection of Pho8p was performed with rabbit anti-Pho8 antibodies at 1:1,000 dilution and of Kar2p with rabbit anti-Kar2 antibodies at 1:1,000 dilution (provided by Dr. R. Piper, University of Iowa, Iowa City, Iowa). The secondary antibody, goat anti-rabbit IgG-horseradish peroxidase (Bio-Rad Laboratories) was used at 1:5,000 dilution.

Vacuole isolation on a Ficoll gradient. Yeast vacuoles were isolated as described previously on discontinuous Ficoll gradients (Conradt et al., 1992). Equal micrograms of vacuoles (found as a band at the interface between the 4% and 0% Ficoll) from each strain were separated on a 7.5% SDS polyacrylamide gel, transferred to nitrocellulose, and immunoblotted as described above.

Extraction of Vac14p from the membrane. Vacuoles from wild-type (LWY7235) cells were diluted to 1 μg/μl with 0% Ficoll buffer (10 mM Pipes, pH 6.8, 200 mM Sorbitol) and 100 μl aliquots were treated with one of the following conditions: 2% Triton X-100, 1.4 M urea, 0.1 M Na2CO3, 1 M NaCl, 0.8 M NH2OH, or left untreated (buffer added to the appropriate volume). Samples were incubated on ice for 30 min and centrifuged at 100,000 g at 4° C. for 30 min and the pellets were resuspended with an equal volume as the corresponding supernatant fraction. Equal amounts were analyzed by SDS-PAGE and immunoblotting as described above.

Analysis of GFP-Fab1p in vivo. GFP was inserted into the FAB1 ORF in pRS426 by homologous recombination. To introduce FAB1 sequences at both the 5′ and 3′ ends, GFP was amplified by PCR from pGOGFP (Cowles et al., 1997) using the primers FABGFP-N (5′-GCT CAC ATG TCC GGT CGTCCT CCA CTG GTA CTT CAT CTG TGA TGG GTA AAG GAG AAG AAC TTT TC-3′) and FABGFPC2 (5′-GCG ACG CAG TGC CGG TCA CGT GAC TTG TTG ATG TCG CTG TTG CGG ATC CCG GGC CCG CGG TAC CGT C-3′). The PCR product and AatII-linearized pRS426-FAB1 were cotransformed into yeast and resultant colonies were screened for GFP fluorescence. The pRS426-GFP-FAB1 plasmid was then transformed into various yeast strains and fluorescence was visualized with the same filter set used for quinacrine.

Analysis of phosphatidylinositols. Cells were grown in YEPD or synthetic media to an OD600 between 0.6 and 0.8. 1 OD600 U of cells was harvested, washed, and resuspended in synthetic media lacking inositol. 0.14-0.4 OD600 U were used to inoculate 5 ml of media lacking inositol containing 50 μCi of myo-[2-³H]-inositol. Cells were labeled for 12 h at 24° C., harvested by centrifugation, washed, and resuspended in 100 μl of inositol-free media. For hyperosmotic shock, an equal volume of 1.8 M NaCl was added to cells (for a final concentration of 0.9 M NaCl) and the resulting suspension was incubated at 24° C. for 10 min. Then, 800 μl of ice cold 4.5% perchloric acid (Whiteford et al., 1996) was add to the cells and transferred to 15 ml Falcon tubes containing 0.5 g acid-washed glass beads. Cells were lysed by vortexing on high for 30 s with at least a 30 s rest (repeated 10 times for each). The cell extracts were centrifuged at 14,000 rpm for 10 min at 4° C. The pellets were washed with 1 ml of 100 mM EDTA, centrifuged as described above, and resuspended in 50 μl of sterile distilled deionized water.

The lipids were deacylated by treatment with methylamine (Hawkins et al., 1986). Briefly, 1 ml of methylamine reagent (10.7% methylamine, 45.7% methanol, 11.4% n-butanol) was added to each and incubated in a 55° C. heat block for 1 h. The samples were dried in a SpeedVac and the pellets were resuspended in 300 μl of sterile water. The samples were centrifuged at 14,000 rpm for 2 min and the supernatants were transferred to new Eppendorf tubes. 300 μl of butanol/ethyl ether/formic acid ethyl ester (20:4:1) was added to each. The samples were vortexed and centrifuged at 14,000 rpm for 2 min. The aqueous phase (bottom layer) was transferred to new tubes and the extraction was repeated. At the end of the second extraction the aqueous phase was dried in a SpeedVac. Samples were resuspended in 20 μl of sterile water and 10 μl of each was analyzed by HPLC using an anion exchange, PartisphereSAX (Whatman), column. The column was developed with a gradient of 1 M (NH4)₂HPO4, pH 3.8 (pH adjusted with phosphoric acid): 1% for 5 min, 1-20% over 44 min, 20-50% over 3.75 min, and remained at 50% for 8 min; the flow rate used was 1.0 ml/min (Schu et al., 1993; Stack et al., 1995).

For comparison of phosphatidylinositol polyphosphate levels, the raw cpms in each peak were expressed as a percentage of the total cpms eluted (phosphatidylinositol is about 90% of the total 3H phosphatidylinositols, whereas phosphatidylinositol polyphosphates are a small percentage, 0.1-3%). The level of PtdIns(3)P identified in the absence of osmotic stress was assigned a value of 100 U; the levels of the other phosphatidylinositol polyphosphates were expressed relative to this value.

### Example 2

### Results

Identification of the VAC14 gene. The Class III vac mutant, vac14-1, was isolated via fluorescence-activated cell sorting (for description of approach see Wang et al., 1996). Like vac7 and fab1 mutants, vac14-1 cells are defective in vacuole inheritance, acidification, and morphology. These mutants have a single, unlobed, enlarged vacuole. Frequently, the vacuole spans both the mother and daughter cell resulting in an “open figure eight” vacuole morphology (Bonangelino et al., 1997).

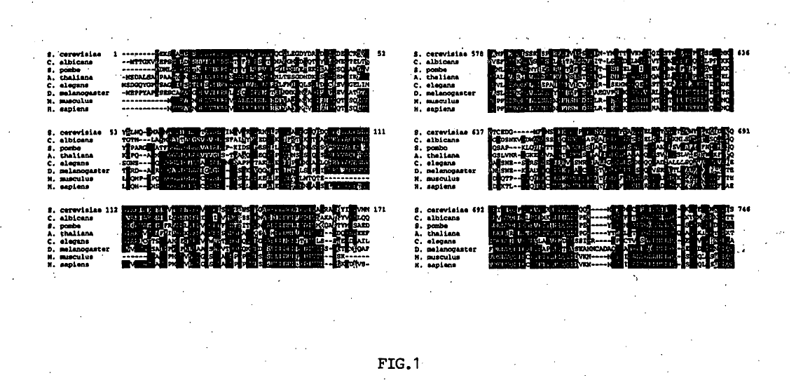

The inventor determined (see Materials and methods) that the VAC14 open reading frame is YLR386W (sequence deposited by the Yeast Genome Sequencing Project). The VAC14 2.64-kb open reading frame encodes a novel polypeptide of 880 amino acids. There are no notable motifs except for a putative transmembrane domain (see below). However, VAC14 displays a high degree of identity with open reading frames present in other eukaryotic organisms. The two regions of highest identity are near the NH2 terminus (residues 1-171) (FIG. 1, left) and COOH terminus (residues 578-746) (FIG. 1, right). Both mouse and human sequences matching either end were identified in the corresponding EST databases. Moreover, a human hypothetical protein, FLJ10305, found on chromosome 16, shows a high degree of identity with the COOH-terminal region of Vac14p and its sequence matches human EST sequences. Both the NH- and COOH-terminal ESTs map to human chromosome 16, suggesting that they correspond to the same gene. No obvious Vac14p homologues were found in any of the published bacterial genomes.

Chromosomal deletion of VAC14. A heterozygous diploid containing a chromosomal deletion of VAC14 was created by replacing the VAC14 open reading frame with TRP1. The resulting diploid was sporulated. In all 20 tetrads examined the TRP1 gene cosegregated with the vac14 phenotypes. vac14-Δ1 cells are viable and, like vac14-1, they display abnormal vacuole morphology, are defective in vacuole inheritance, and have a vacuole acidification defect. vac14-1 and vac14-Δ1 cells have nearly identical growth rates, with a doubling time that is similar to that of wild-type cells (unpublished data). Because vac14-1 and vac14-Δ1 behave identically in all tests performed, it is likely that the original vac14-1 allele is a loss of function mutation.

The growth defects of the double mutants, vac7-1, vac14Δ and fab1-Δ1, vac14Δ were similar to vac7-Δ1, or fab1-Δ1 mutants alone, consistent with VAC14, VAC7, and FAB1 functioning in the same pathway. The inventor was unable to determine whether VAC7 functions upstream or downstream of FAB1 or VAC14 because VAC7 expressed from either a low-copy or multi-copy plasmid did not suppress the vac14-1, vac14-Δ1 or fab1-Δ1 mutant phenotypes (Bonangelino et al., 1997), and neither FAB1 nor VAC14 suppressed vac7-Δ1. However, a similar analysis suggested that VAC14 functions upstream of FAB1. Overexpression of FAB1 suppressed the vacuolar morphology, inheritance and acidification defects of the vac14-Δ1 mutant, and the vac14-1 mutant (Bonangelino et al., 1997). This suppression required the kinase activity of Fab1p since overexpression of a kinase-dead fab1-D2134R mutant (Gary et al., 1998) did not suppress the vac14-Δ1 mutant phenotypes (unpublished data). These data suggest that Vac14p may be an activator of Fab1p kinase activity.

Vac14p associates with vacuolar membranes. Antibodies raised to a glutathione S-transferase (GST)—Vac14p fusion protein recognized a polypeptide with a molecular weight of approximately 99 kD, the predicted molecular weight of Vac14p (A second, 70-kD polypeptide is nonspecific as it appears in both wild-type and vac14-Δ1 extracts). The majority of Vac14p was found in a membrane-associated fraction that pelleted at 13,000 g (P13). The P13 fraction contains large organelles such as vacuoles, ER, and nuclei. A similar fractionation pattern was observed for other vacuolar proteins, such as Vac8p (Wang et al., 1998).

Vac14p was present on vacuoles isolated on a Ficoll flotation gradient and was enriched on purified vacuoles to a similar extent as the vacuolar membrane protein, Pho8p. Sequence analysis of Vac14p revealed a putative transmembrane domain between residues 430 and 451 (determined by PSORT and hydropathy plot analysis). Despite this prediction, Vac14p was peripherally associated with vacuole membranes. Treatments with either 0.1 M Na2CO3, pH 11.5, 1 M NaCl, or 0.8 M NH2OH-solubilized Vac14p, whereas Pho8p was not extracted by any of these treatments.

Vac14p was also present in vacuoles isolated from vac7-Δ1 and fab1-Δ1 strains. Thus, defects observed in vac7-Δ1 cells are not due to a gross mislocalization of Vac14p. Interestingly, although fab1-Δ1 cell extracts had normal levels of Vac14p, the vacuoles contained about 50% less Vac14p than wild-type vacuoles. The decrease of Vac14p on fab1-Δ1 vacuoles was observed in four independent experiments and suggests that Vac14p may physically associate with Fab1p. Since some Vac14p remained associated with vacuoles in the absence of Fab1p, there are likely to be additional proteins that bind Vac14p and help mediate its association with vacuolar membranes. Vac7p is not a candidate because vac7-Δ1 vacuoles contain normal levels of Vac14p. The inventor was unable to detect a direct physical interaction between Vac14p and Fab1p either by yeast two-hybrid, coimmunoprecipitation, or cross-linking experiments. Thus, any interaction is likely to be transient or indirect, and therefore difficult to detect by standard biochemical techniques. Cellular fractionation alone was used to determine Vac14p localization because the inventor was unable to use the goat serum for indirect immunofluorescence.

Proper GFP-Fab1p localization does not depend on Vac7p or Vac14p. Fab1p, like Vac14p, is peripherally associated with vacuolar membranes (Gary et al., 1998). A GFP-Fab1p fusion protein was constructed to visualize Fab1p in various mutant strains. The GFP-Fab1p localized to endosomal and vacuolar membranes in wild-type yeast, fab1-Δ1, vac7-Δ1, and vac14-Δ1 mutants. This demonstrates that defects in PtdIns(3,5)P2 levels in vac7-Δ1 and vac14-Δ1 mutants are not due to Fab1p mislocalization. Moreover, both Vac14p and Fab1p are localized on the vacuole, consistent with Vac14p serving as an activator of Fab1p.

An improved method to quantify phosphatidylinositol polyphosphate levels in yeast. Standard methods for analyzing phosphatidylinositol levels in yeast have consisted of lysing cells in acidified chloroform/methanol/HCl with the concomitant extraction of total cellular lipids. The inventor tested this method on wild-type yeast in the absence and presence of osmotic shock and obtained phosphatidylinositol polyphosphate levels similar to those reported previously (Dove et al., 1997; Gary et al., 1998). However, the inventor also noted that there were substantial fluctuations in the levels of Ptd-Ins(3,5)P2 and PtdIns(4,5)P2 even in identically prepared samples. Therefore, the inventor adapted a method described for analyzing PtdIns(3,5)P2 levels in tissue culture cells, where cells are lysed in 4.5% perchloric acid (Whiteford et al., 1997). This treatment precipitates most macromolecules, including proteins and lipids. The lipids are then deacylated with methylamine and the released glycero-phospho-inositols are extracted with water. Notably, the inventor obtained significantly higher quantities of the corresponding inositol monophosphates and biphosphates with far less variability among identically prepared samples for the inositol bisphosphates. This allowed us to reproducibly detect basal levels of PtdIns(3,5)P2, which are about 15-fold higher than reported previously.

PtdIns(3,5)P2 levels rise 20-fold in response to osmotic stress. Using this improved method the inventor found that osmotic stress results in much higher levels of PtdIns(3,5)P2 than reported previously. 10 min after exposure to osmotic stress, the levels of PtdIns(3,5)P2 and Ptd-Ins(4,5)P2 are similar to PtdIns(3)P and PtdIns(4)P levels. Although the levels of each of the phosphorylated phosphatidylinositols underwent some change after exposure of the cells to osmotic stress, the change in Ptd-Ins(3,5)P2 levels was the most dramatic, with the levels of this lipid rising 16-20-fold. Quantification of the fold increase could not be performed on cells extracted with chloroform methanol HCl because with this method PtdIns(3,5)P2 was not detectable in cells grown in the absence of osmotic shock. Moreover, after a 10-min exposure to osmotic stress, approximately 8-fold more PtdIns(3,5)P2 and 7-fold more PtdIns(4,5)P2 were detected in cells lysed with perchloric acid compared with cells extracted with chloroform methanol HCL.

Fab1p, Vac7p, and Vac14p are required for steadystate levels of PtdIns(3,5)P2 during vegetative growth. The inventor next reevaluated PtdIns(3,5)P2 levels in both vac7-Δ1 and fab1-Δ1. As reported previously (Gary et al., 1998), there was no detectable PtdIns(3,5)P2 in fab1-Δ1, consistent with Fab1p being the sole 5-kinase that converts PtdIns(3)P to PtdIns(3,5)P2. Likewise, no PtdIns(3,5)P2 was detected in the vac7-Δ1 mutant. The inventor also observed an elevation in PtdIns(3)P levels in these mutants following osmotic stress (see below).

The inventors next analyzed PtdIns(3,5)P2 levels in the vac14-Δ1 mutant. Like vac7 and fab1 mutants, in the absence of osmotic stress, no detectable PtdIns(3,5)P2 was observed. These results suggest that the Class III vac mutant phenotypes shared by vac7, fab1, and vac14 mutants are due to a deficiency in PtdIns(3,5)P2 levels.

vac14 mutants synthesize low levels of PtdIns(3,5)P2 after exposure to osmotic stress. Unlike vac7 and fab1 mutants, when vac14-Δ1 cells were exposed to 0.9 M NaCl for 10 min, a small amount of Ptd-Ins(3,5)P2 was observed (1.7 U). This level is similar to the basal levels detected during vegetative growth (3.5 U), but much lower than the osmotic stress-induced levels (71 U) observed in wild-type cells.

The inventors suspect that low, yet undetectable, levels of Ptd-Ins(3,5)P2 are synthesized in vac14-Δ1 cells in the absence of osmotic stress. First, vac14-Δ1 mutants produce Ptd-Ins(3,5)P2 during osmotic shock, demonstrating that Fab1p, the phosphatidylinositol (3)P 5-kinase, retains partial activity in the absence of Vac14p. Second, the doubling time of vac14 cells is similar to that of wild-type cells (unpublished data), in contrast to the greatly reduced growth rate of fab1Δ and vac7Δ (Bonangelino et al., 1997). The nearly normal doubling time of vac14 mutants is likely due to synthesis of small amounts of Ptd-Ins(3,5)P2.

Vac14p is required for elevation of PtdIns(3,5)P2 in response to osmotic stress. Because overexpression of FAB1 suppresses the vacuolar defects of the vac14-Δ1 mutant, the inventor tested Ptd-Ins(3,5)P2 levels in these strains. Expression of FAB1 from a low- or high-copy plasmid in the vac14-Δ1 mutant brought the levels of PtdIns(3,5)P2 from undetectable to nearly the basal levels detected in wild-type cells (2 U). These observations are consistent with Vac14p functioning upstream of Fab1p.

Notably, FAB1 expression in vac14 mutants did not restore the elevation of PtdIns(3,5)P2 normally observed in response to osmotic shock. The levels of PtdIns(3,5)P2 after osmotic shock in vac14-Δ1, or vac14-Δ1 with either a low or high copy FAB1, were statistically identical (2.4, 3.2, and 3.1 U, respectively). As a control, vac14-Δ1 with VAC14 expressed from a low copy plasmid produced 30 U of PtdIns(3,5)P2 in response to osmotic shock. Thus, Vac14p is required for the normal increase of Ptd-Ins(3,5)P2 in response to hyperosmotic stress. The small increase in PtdIns(3,5)P2 observed after osmotic shock in the vac14-1 and vac14-Δ1 mutants is most likely due to an osmotic stress-induced elevation of the biosynthetic precursor, PtdIns(3)P (see below).

Vps34p activity increases in response to osmotic stress. As PtdIns(3,5)P2 levels rise, one would expect to see an equivalent decrease of the precursor, PtdIns(3)P. In fact, after exposure to osmotic stress, the inventor observed a decrease in Ptd-Ins(3)P in wild-type cells. However, our data suggests that the rate of PtdIns(3)P synthesis is elevated. First, in wild-type cells, the decrease in PtdIns(3)P by 32 U is not enough to account for the 71 U increase in PtdIns(3,5)P2. Second, if the rate of PtdIns(3)P synthesis rises due to osmotic stress, then one would expect to detect this rise in cells that are blocked in PtdIns(3,5)P2 synthesis. Indeed, in fab1-Δ1 and vac7-Δ1 mutants, the levels of Ptd-Ins(3)P after exposure to osmotic stress increased by 35 and 40 U, respectively. This suggests that after exposure to osmotic stress, PtdIns(3)P levels increase and are quickly depleted by the rapid production of PtdIns(3,5)P2. However, in mutants that cannot synthesize PtdIns(3,5)P2, the extra PtdIns(3)P accumulates.

In wild-type cells, the elevation of PtdIns(3)P in response to osmotic stress likely compensates for the rapid conversion of PtdIns(3)P to PtdIns(3,5)P2. Lack of a compensatory mechanism would deplete the levels of PtdIns(3)P, which are required for membrane trafficking to the yeast vacuole (Schuet al., 1993). In addition, a rise in PtdIns(3)P in response to osmotic shock might also be responsible for producing an increase in PtdIns(3,5)P2, as PtdIns(3)P could directly activate Fab1p or drive the reaction forward by mass action.

Although it is possible that Vac14p might indirectly affect PtdIns(3,5)P2 levels by regulating Vps34p activity, our data demonstrate that this is not the case. If Vac14p activated Vps34p, then in a vac14-Δ1, fab1-Δ1 double knockout there would be little or no PtdIns(3)P accumulation. However, in a vac14-Δ1, fab1-Δ1 double mutant, the levels of PtdIns(3)P were virtually identical to fab1-Δ1 mutants, with a large accumulation of PtdIns(3)P in the presence of osmotic stress (an elevation of 40 and 35 U, respectively). Thus, while osmotic stress activates Vps34p, Vac14p is not involved in this activation. Moreover, the defects in PtdIns(3,5)P2 levels in vac14-Δ1 cells are not indirectly due to reduced PtdIns(3)P production.

Vac14p is an osmotic response activator of Fab1p. The observation that during osmotic stress vac14 mutants have low levels of PtdIns(3,5)P2 and accumulate PtdIns(3)P suggests either of two remaining hypotheses. First, Vac14p may function as an osmotic response activator of Fab1p. In the absence of its activator, Fab1p functions with reduced activity, leading to the accumulation of its substrate, Ptd-Ins(3)P. Alternatively, Vac14p may negatively regulate a phosphatidylinositol 5-phosphatase required for Ptd-Ins(3,5)P2 turnover which would also result in accumulation of PtdIns(3)P. Although turnover of PtdIns(3,5)P2 might also be achieved through the actions of a phosphatidylinositol 3-phosphatase, a polyphosphatidylinositol phosphatase, and/or lipases, none of these would result in the observed accumulation of PtdIns(3)P.

If Fab1p is activated in response to osmotic stress, then there might be a subset of fab1 mutants that produce basal levels of PtdIns(3,5)P2 but are incapable of further activation. The fab1-2 mutant displays this phenotype (Cooke et al., 1998; Gary et al., 1998). At the permissive temperature (24° C.), fab1-2 had normal levels of PtdIns(3,5)P2 during vegetative growth (3.1 U), but after exposure to osmotic shock the elevation in PtdIns(3,5)P2 levels was greatly reduced (12 U) compared with wild-type levels (71 U). In addition, overexpression of Vac14p in the fab1-2 mutant did not increase the levels of PtdIns(3,5)P2 produced in response to osmotic shock (8 U). Moreover, overexpression of Fab1p in fab1-2 did not fully restore levels of PtdIns(3,5)P2 to those seen in wild-type cells (27 instead of 71 U). This suggests that fab1-2 is incapable of full activation by Vac14p and is partially dominant, perhaps by sequestering Vac14p. Consistent with this hypothesis, simultaneous overexpression of wild-type Fab1p and Vac14p in fab1-2 resulted in much higher levels of PtdIns(3,5)P2 (40 U).

If Vac14p was the inhibitor of the phosphatidylinositol 5-phosphatase, an increase in PtdIns(3,5)P2 would be expected when VAC14 is overexpressed in fab1-2 cells. In fact, overexpression of VAC14 did not elevate the levels of Ptd-Ins(3,5)P2 in this strain. Since Vac14p is already present, it is possible that the phosphatidylinositol 5-phosphatase is fully inhibited and thus adding more Vac14p has no effect. However, if this were true, simultaneous overexpression of Vac14p and Fab1p should produce the same levels of PtdIns(3,5)P2 as overexpression of Fab1p alone. The inventor observed that simultaneous overexpression of Vac14p and Fab1p in fab1-2 resulted in 48% more PtdIns(3,5)P2 than in fab1-2 overexpressing only Fab1p. These results strongly suggest that Fab1p and Vac14p act in concert with each other and are consistent with Vac14p functioning as an activator of Fab1p rather than as the negative regulator of a phosphatidylinositol 5-phosphatase.

Levels of PtdIns(3,5)P2 regulate vacuolar morphology. Observations of the dramatic increase in PtdIns(3,5)P2 levels combined with our identification of a molecule required for activation of Fab1p strongly suggest that PtdIns(3,5)P2 protects yeast from rapid increases in osmolarity. As described below, it is likely that this protection occurs via Ptd-Ins(3,5)P2 regulation of vacuole volume. Overproduction of PtdIns(3,5)P2 resulted in both an increase in the number and a decrease in size of vacuole lobes. In this case, elevation of PtdIns(3,5)P2 was achieved in the absence of osmotic stress by simultaneous overexpression of both Vac14p and Vac7p. This resulted in a 70% increase in basal levels of Ptd-Ins(3,5)P2, 12 U, instead of 7 U (wild-type expressing the vectors alone). Conversely, a pronounced phenotype in cells with defects in steady-state levels of PtdIns(3,5)P2 is an enlarged unlobed vacuole. These observations suggest that Ptd-Ins(3,5)P2 levels regulate the surface area to volume ratio of the vacuole by changing the number and size of the vacuole lobes. Note that a sphere of a given diameter can create four spheres with a combined total of half the volume using the same total membrane surface area. Thus, increasing Ptd-Ins(3,5)P2 levels in response to osmotic stress may serve to regulate vacuole morphology under conditions when the vacuole loses volume due to loss of water. Consistent with this hypothesis, the inventor found that after a short exposure to hyperosmotic stress, the vacuole morphology of wild-type cells changed to smaller, more highly fragmented vacuoles. However, the vacuole morphology of mutants unable to produce PtdIns(3,5)P2 remained largely unchanged. This demonstrates that changes in vacuole morphology induced by exposure to osmotic stress are regulated via a molecular mechanism that requires the synthesis of more Ptd-Ins(3,5)P2. Moreover, this observation provides the first insight into a physiological consequence of increased Ptd-Ins(3,5)P2 production in response to osmotic stress.

Vacuole fission and tubulation. Membrane deformation occurs when vesicles and tubules bud and subsequently pinch off from a larger organelle. How this occurs is a central question in cell biology. The discovery and analysis by the inventor of the Class III vac mutants indicates that there is an underlying commonality to all these types of membrane deformation and deomonstrate that PPIs play a key role.

Class III vac mutants are defective in PI3,5P2 synthesis. Class III vac mutants were isolated based on their vacuole inheritance defect and found that they are defective in several processes that involve deformation of the vacuole membrane. These processes include an inability to form segregation structures (Bonangelino et al., 1997), a defect in vacuole fission (Bonangelino et al., 1997), and a defect in retrograde traffic from the vacuole57. Three complementation groups with these phenotypes were identified, fab1, vac7 and vac14. The inventor subsequently discovered that each of the corresponding proteins Fab1p, Vac7p and Vac14p, are required to maintain normal levels of PI3,5P2 (Gary et al., 1998).

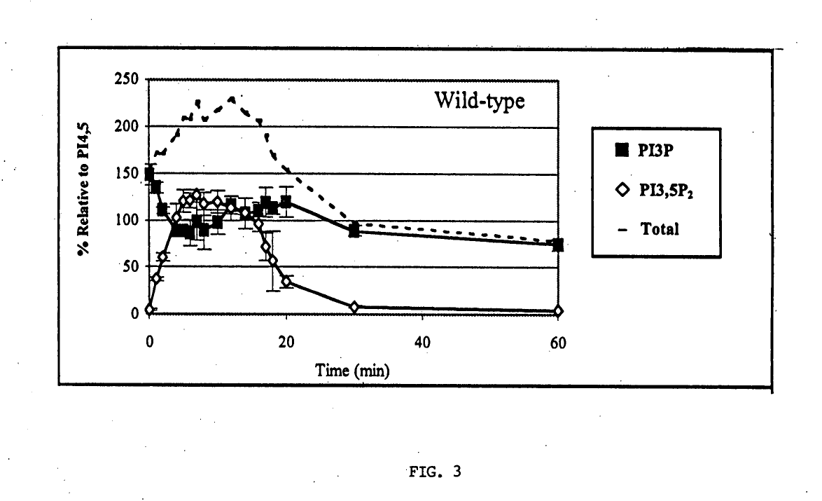

Osmotic stress is an external stimulus that transiently increases the cellular levels of PI3,5P2. In yeast, the cellular levels of PI3,5P2 are 18-28 fold lower than the other yeast phosphoinositides, PI3P, PI4P, and P14,52, but rise dramatically in response to osmotic stress (Dove et al., 1997). The inventor developed a method that reproducibly extracts phosphoinositides from yeast. In all published studies examining the effect of osmotic stress on PI3,5P2, including those of the inventor, only two time points were assayed, zero time (no osmotic stress) and 10 min after addition of osmolyte. In order to obtain a more detailed view of the osmotic stress induced changes on PI3,5P2 levels, several time points were assayed and it was discovered that within minutes of exposure to hyperosmotic stress, PI3,5P2 levels rise over 20-fold (FIG. 3 and Table 2). This brings PI3,5P2 to a cellular concentration that is similar to other phosphoinositide species. By 30 min its cellular concentration returns to basal levels.

TABLE 1
Class III vac Mutants Fail to Maintain Normal Levels of P1(3,5)P₂

| | PI3P | | PI3,5P₂ | |
|---|---|---|---|---|
| 0.9 M NaCl | − | + | − | + |
| LWY7235 | 149 ± 11 | 98 ± 13 | 5 ± 1 | 120 ± 12 |
| Δfab1 | 126 ± 13 | 170 ± 9 | <1 | <1 |
| Δvac7 | 147 ± 12 | 201 ± 9 | <1 | <1 |
| Δvac14 | 135 ± 18 | 181 ± 15 | <1 | 2 ± 0 |
| Δfig4 | 158 ± 12 | 198 ± 16 | 5 ± 1 | 23 ± 0 |
| Δfab1Δvac14 | 147 ± 5 | 205 ± 7 | <1 | <1 |

Phosphoinositide levels are reported relative to P14,5P₂ levels in a wild-type strain under basal conditions (100 units). The data (mean±standard deviation) were obtained from a miniumum of four independent studies.

The transient rise in PI3,5P2 levels (FIG. 3) is accompanied by a transient reduction in PI3P levels (FIG. 3), however, the overall rate of accumulation of PI3P rises, as indicated by the rise in PI3P+PI3,5P2 (FIG. 3). Note that the subsequent rapid disappearance of the PI3P+PI3,5P2 pool is mostly due to the disappearance of PI3,5P2. This transient change in PI3,5P2 levels directly corresponds with transient changes in vacuole morphology. Higher levels of PI3,5P2 correlate with a lower vacuole volume. This correlation holds true even in the absence of osmotic stress. When PI3,5P2 levels are raised by overexpression of the Fab1p regulators, Vac7p and Vac14p, the number of vacuole lobes is higher, while the size of each lobe is smaller.

The elevation in PI3,5P2 may prevent vacuole lysis by simultaneously regulating vacuole ion influx/efflux, water efflux, and vacuole volume. PI3,5P2 regulation of vacuole volume occurs via at least two membrane trafficking pathways, retrograde traffic from the vacuole to late endosome and vacuole fission. Thus, the inventors have identified a situation where a stimulus outside of the cell, osmotic stress, produces an almost immediate change in the level of a single type of lipid that resides late in the endomembrane system. Since this dramatic change can be triggered in an entire population of cells, it is now possible to (1) determine how PI3,5P2 synthesis and turnover is regulated, (2) determine the pathways that are regulated by PI3,5P2 and (3) study how PI3,5P2 regulates each process.

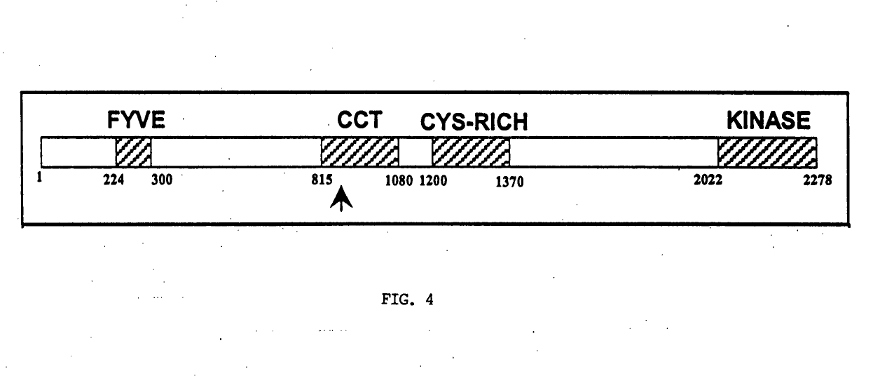

Characterization of the PI3P 5-kinase Fab1p. Fab1p is the only PI3P 5-kinase in yeast (Gary et al., 1998). Based on the sequence of FAB1, it was predicted that Fab1p encodes a lipid kinase. In collaboration with S. Emr and coworkers, the inventor tested the levels of all the PPIs in a fab1 strain and found that no PI3,5P2 is produced (Gary et al., 1998). Subsequently a more sensitive method for detection of these lipids has been developed which confirmed that synthesis of PI3,5P2 requires Fab1p. However, overexpression of Fab1p alone does not increase PI3,5P2 levels, suggesting that the regulation of Fab1p is complex. Indeed Fab1p is an extremely large protein—2278 amino acids—which is approximately four-fold longer than other known phosphatidylinositol 5-kinases. There are at least two potential regulatory domains in Fab1p (FIG. 4), a FYVE domain which has been shown to bind PI3P, and the CCT domain, which has homology to a region of a regulatory subunit of the groEL family of chaperonins.

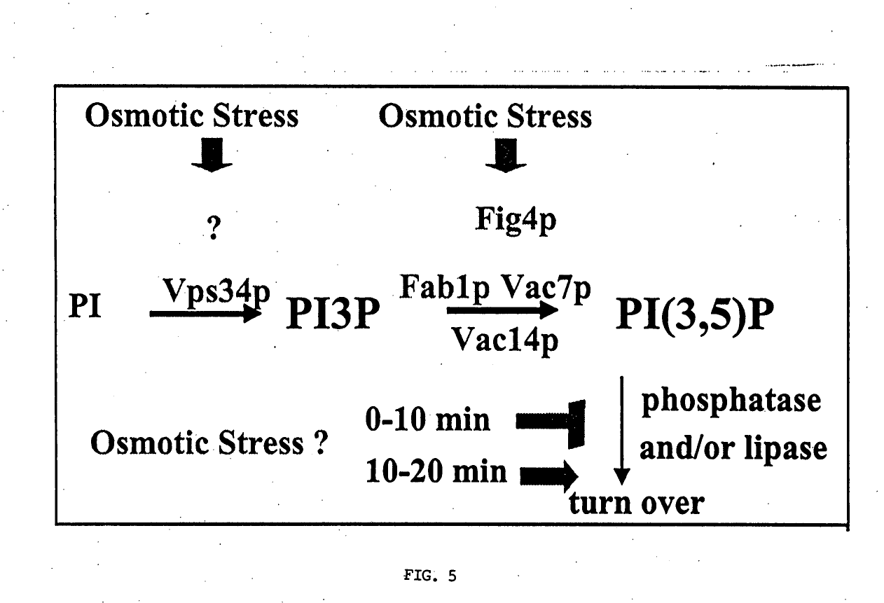

The inventor has discovered three proteins that activate Fab1p: Vac7p, Vac14p and Fig4p. The inventor's current model is that Vac7p and Vac14p activate Fab1p under basal conditions, but Fig4p is not required. During osmotic stress, Vac7p, Vac14p and Fig4p are required. Vac14p and Fig4p interact by two-hybrid and may form a complex. The working model is shown in FIG. 5.

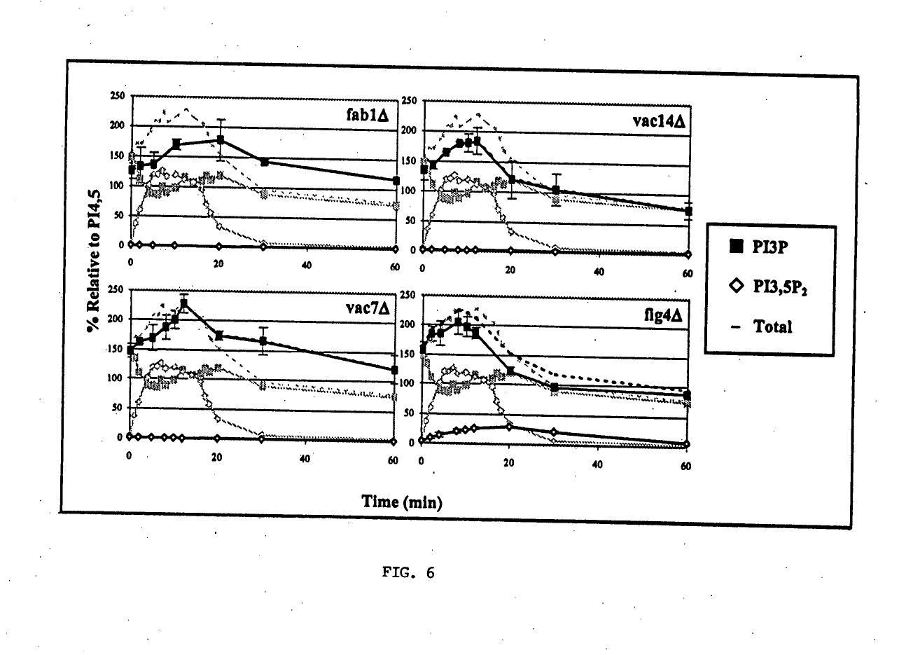

Vac7p is required for Fab1p function both in the presence and absence of osmotic stress. The phenotypes of both vac7 and fab1 are virtually identical, no PI3,5P2 is detected in either strain (FIG. 6; Table 2). However, the inventor identified an activated fab1 mutant that bypasses the need for Vac7p (Gary et al., 2002), suggesting that Vac7p functions in a regulatory role and that the enzymatic activity resides in Fab1p.

Vac14p is also required for Fab1p activity, both in the presence and absence of osmotic stress, however it is particularly important during osmotic stress. What is most notable about the vac14 mutant, is the lack of the dramatic rise in PI3,5P2 levels during osmotic stress (FIG. 6; Table 2). Although it could not be detected by our methods, it is likely that some PI3,5P2 is synthesized under basal conditions in vac14.

Further support for Vac14p as an activator of Fab1p is the analysis of fab1-2, which is specifically defective in being activated during osmotic stress and does not respond to Vac14p. The inventor has now determined that the fab1-2 defect is caused by a single point mutation in the CCT domain (FIG. 4). The function of this domain is unknown, but it is conserved in all Fab1p orthologues. The inventor is currently testing whether Vac14p interacts with this domain, and also whether overexpression of this domain alone has a dominant-negative effect.

The data described here suggest that Vac14p functions together with Vac7p. Overexpression of Vac14p alone does not affect PI3,5P2 levels, while the combined overexpression of Vac7p and Vac14p raises the levels of PI3,5P2.

Fig4p is required for the osmotic stress induced activation of Fab1p. Fig4p is one of five proteins in yeast that contains a Sac1-domain (Guo et al., 1999). Three of the other SAC1 domain containing proteins Sac1p, Sjl2/Inp52 and Sjl3/Inp (Wang et al., 2002) have been demonstrated to function as lipid phosphatases and are able to dephosphorylate PI3,5P2, PI3P, and PI4P in vitro (Guo et al., 1999; Hughes et al., 2000). In a recent study with S. Emr and coworkers, the inventor suggested that Fig4p is a lipid phosphatase that acts on PI3,5P2 (Gary et al., 2002).

Reinterpretation of the data along with new data suggests that Fig4p activates Fab1p. The inventor identified fig4-1 as a genomic suppressor of vac7. A fig4-1, vac7 mutant produces near wild-type levels of PI3,5P2. Moreover fig4-1 on its own has 3-fold higher levels of PI3,5P2. Conversely, overexpression of Fig4p lowers PI3,5P2 levels. These phenotypes are consistent with FIG4 encoding a PI3,5P2 phosphatase. However, fig4-1 is not a null mutant, because PI3,5P2 levels are normal in fig4. These latter data indicate that Fig4p is not a PI3,5P2 phosphatase. Fig4p is required for Fab1p activation during osmotic stress, but is not required for maintaining basal levels of PI3,5P2 (FIG. 6; Table 2). It is also difficult to reconcile this last result with the hypothesis that Fig4p functions as a PI3,5P2 phosphatase. Thus, it is hypothesized that Fig4p interacts with Vac14p to activate Fab1p. Therefore, the inventor's current hypothesis of fig4-1 is that it is a mutant that constitutively activates Fab1p. Thus, Fig4p is believed to be a protein phosphatase that dephosphorylates Fab1p.

Neither Fig4p nor Vac14p activate Vps34p. Another way that PI3,5P2 levels could be controlled is via regulation of the levels of the precursor, PI3P. However, Fig4p and Vac14p activate Fab1p and do not appear to regulate Vps34p because in a vac14 or fig4 strain the kinetics of the rise of PI3P levels is the same as the wild-type rise in PI3P+PI3,5P2 (FIG. 6).

The activation of Fab1p plays a key role in the rapid rise in PI3,5P2 levels after osmotic shock. The steady-state levels of any macromolecule are regulated by its rate of synthesis, rate of turnover, or a combination of both. The osmotic stress induced rise in PI3,5P2 levels is regulated by activation of Fab1p. Yeast which express mouse PIKfyve/Fab1 as the sole copy of Fab1 synthesize basal levels of PI3,5P2, but fail to achieve elevated levels of PI3,5P225. Note that the enzymes required for turnover are present in that strain. While negative regulation of the enzymes required for PI3,5P2 turnover do not appear to be required in the initial response to osmotic stress, the rapid disappearance of PI3,5P2 10-20 min after osmotic stress could be due to inactivation of Fab1p and/or changes in the rate of turnover.

The turnover of the PI3,5P2 produced in response to osmotic stress does not require Fig4p or Vac14p. The kinetics of the disappearance of PI3P in fig4 and vac14 is the same as the disappearance of PI3P+PI3,5P2 in a wild-type strain (FIG. 6). Moreover, the rate of disappearance of PI3P+PI3,5P2 in wild-type is mostly due to the disappearance of PI3,5P2. These findings indicate that the enzymes that turnover PI3,5P2 can also act on PI3P, and do not require Vac14p or Fig4p.

PI3,5P2 is required for retrograde traffic from the vacuole to the late endosome. In collaboration with Rob Piper (U. of Iowa) and Nia Bryant and Tom Stevens (U. of Oregon), the inventor found that vac7 is defective in a novel membrane trafficking pathway, movement of vacuole resident proteins back to the Golgi. The inventor now finds that fab1 and vac14 are also defective in this pathway, demonstrating that it is the lack of PI3,5P2 that causes the defect. This defect could account for the grossly enlarged vacuoles seen in these mutants. Note that only approximately half the GFP-RS-ALP has moved from the Golgi to the vacuole.

It had previously been reported that PI3,5P2 is also required for formation of multivesicular bodies (MVB) (Odorizzi et al., 1998). It now appears that this is not the case. PI3,5P2 may be required for targeting selected proteins into the MVB (Urbanowski et al., 2001; Reggiori and Pelham, 2002).

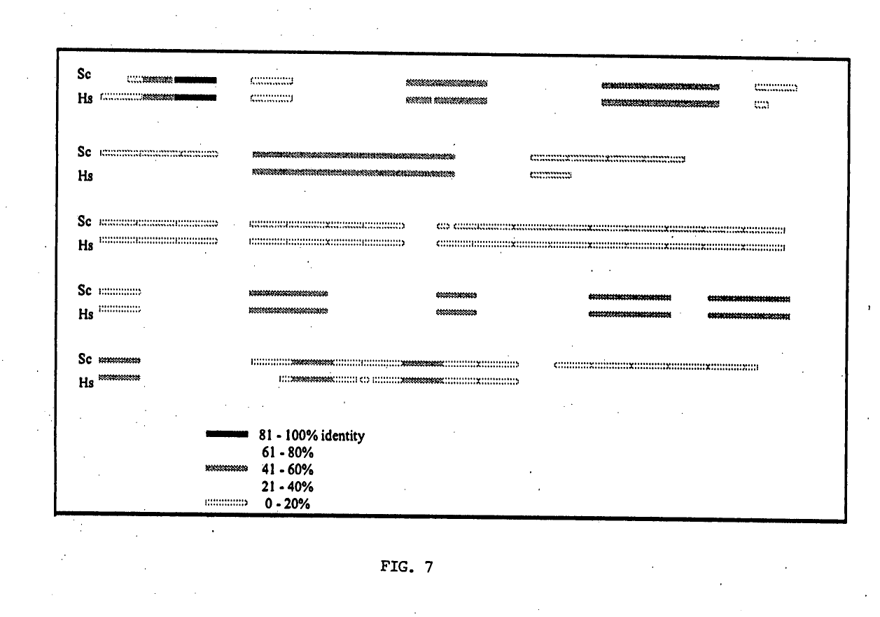

Identification and sequence analysis of human orthologues of Fab1p, Vac14p and Fig4p. Because PI3,5P2 has been found in all eukaryotes tested, the inventor performed BLAST searches with Fab1p, Vac7p, Vac14p and Fig4p to determine whether these proteins had homologues in other species. The inventor was particularly interested in identification of human ESTs which potentially represented cDNAs that encode proteins with high sequence similarity to these three yeast proteins. Corresponding IMAGE clones were obtained and it was found that each contained partial cDNA sequences. Primers were designed to PCR amplify the missing regions. A human adipocyte cDNA pool was used for these amplifications because transcripts for mouse FAB1/PIKfyve were found to be elevated in differentiated 3T3-L1 adipocytes (Shisheva et al., 1999). A full-length cDNA was obtained and sequenced for each of hFab1, hVac14 and hFig4. Both hVac14 and hFig4 show-significant sequence similarity to Vac14p and Fig4p, respectively (FIG. 7).

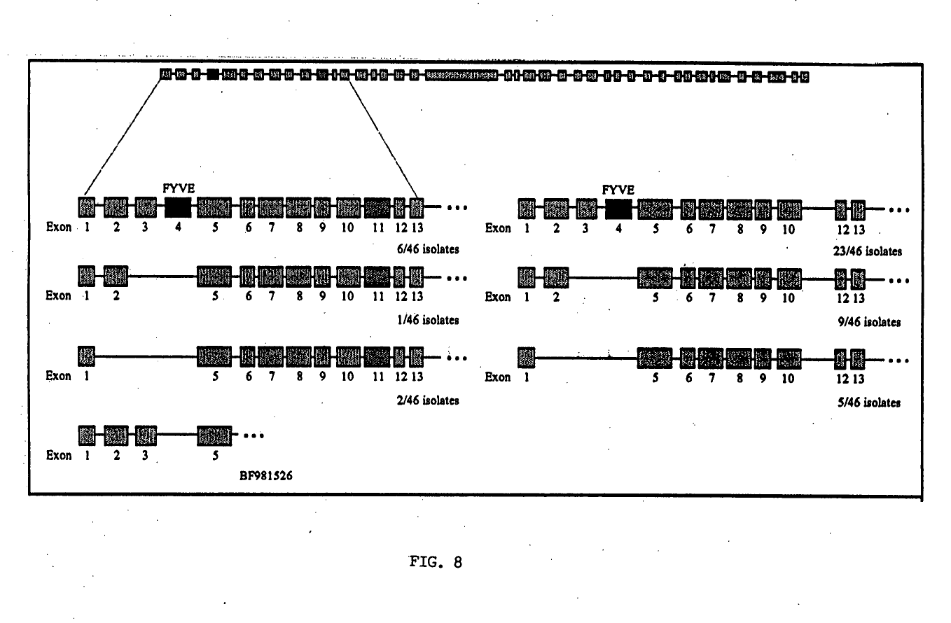

Notably, the inventor obtained several cDNAs for hFab1. The sequences indicate that each cDNA came from the same gene and that the differences arose from alternative splicing. The alternative splice sites were confined to the 5′ end of the gene. The hFab1 gene is large and spans approximately 40 kb and contains 41 exons (FIG. 8). Some splice variants included cDNAs with and without exon 11 (SEQ ID NOS:9 and 10). Exon 11 may be human-specific, because exon 11 was not present in the published sequence of mouse PIKfyve. A second set (30%) of the hFab1 isoforms were missing exon 4, which encodes the FYVE domain (FIG. 8). The FYVE domain binds PI3P, and is conserved in all species examined.

hFab1 and hVac14 reside in an endosomal compartment, a localization consistent with a potential signaling role at a GLUT4-specific compartment. A polyclonal rabbit anti-serum raised against a fusion protein of GST and the first 100 amino acid residues of hFab1 specifically recognizes human Fab1. Using Western analysis, a protein of the expected molecular weight, approximately 240,000 daltons, was detected in mammalian tissue culture cell lysates. Similarly, a polyclonal sheep anti-serum raised against a fusion of GST and full-length human Vac14, specifically recognizes human Vac14, and reveals a protein of the expected molecular weight, 90,000 daltons. These reagents were used by the inventor to test the localization of hFab1 and hVac14. Human Fab1 and human Vac14 co-localize with each other and with EEA1, a well-characterized protein that is known to reside on the early endosome. Co-localization was observed in the two cell-lines tested, Vero cells and NRK cells. Because GLUT4 compartments are derived from early endosomes, the localization of Fab1 and Vac14 to an early endosomal compartment further supports the hypothesis that these proteins may reside in GLUT4 containing compartments in adipocytes and muscle cells.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XII. REFERENCES

The following references, to the extent that they provide exemplary procedural or other details supplementary to those set forth herein, are specifically incorporated herein by reference.
U.S. Pat. No. 4,415,732
U.S. Pat. No. 4,458,066
U.S. Pat. No. 4,554,101
U.S. Pat. No. 4,683,195
U.S. Pat. No. 4,683,202
U.S. Pat. No. 4,800,159
U.S. Pat. No. 4,873,191
U.S. Pat. No. 4,883,750
U.S. Pat. No. 5,279,721
U.S. Pat. No. 5,354,855
U.S. Pat. No. 5,792,453
U.S. Pat. No. 5,795,715
U.S. Pat. No. 5,795,715
U.S. Pat. No. 5,889,136
U.S. Pat. No. 6,100,242
Altschul et al., J. Mol. Biol., 215 (3): 403-410, 1990.
Baichwal et al., In: Gene transfer, Kucherlapati (Ed), Nyork, Plenum Press, 117-148, 1986.
Barany and Merrifield, In: The Peptides, Gross and Meienhofer (Eds.), Academic Press, New York, 1-284, 1979.
Benvenisty and Neshif, Proc. Natl. Acad. Sci. USA, 83 (24): 9551-9555, 1986.
Bonangelino et al., Mol. Cell Biol., 17 (12): 6847-6858, 1997.
Bosher and Labouesse, Nat. Cell Biol., 2: E31-E36, 2000.
Brinster et al., Proc. Natl. Acad. Sci. USA, 82 (13): 4438-4442, 1985.
Bryant et al., J. Cell Biol., 142 (3): 651-653, 1998.
Cantley and Neel, Proc. Natl. Acad. Sci. USA, 96 (8): 4240-4245, 1999.
Capaldi et al., Biochem. Biophys. Res. Comm., 74 (2): 425-433, 1977.
Caplen et al., Gene, 252 (1-2): 95-105, 2000.
Chang et al., Hepatology, 14: 134A, 1991.
Chen and Okayama, Mol. Cell Biol., 7: 2745-2752, 1987.
Chen et al., J. Biol. Chem., 272 (43): 27401-27410, 1997.
Coffin, In: Virology, Fields et al. (Eds.), NY, Raven Press, 1437-1500, 1990.
Conradt et al., J. Cell Biol., 119 (6): 1469-1479, 1992.
Cooke et al., Curr. Biol., 8 (22): 1219-1222, 1998.
Couch et al., Am. Rev. Resp. Dis., 88: 394-403, 1963.
Coupar et al., Gene, 68: 1-10, 1988.
Cowles et al., Cell, 91 (1): 109-118, 1997.
Cremona and De Camilli, J. Cell Sci., 114 (Pt6): 1041-1052, 2001.
Cullen et al., Curr. Biol., 11 (21): R882-893, 2001.
Culliton, Science 246: 746-751, 1989.
Davis et al., Meth. Enzymol., 65: 404-411, 1980.
Dove et al., Curr. Biol., 12 (11): 885-893, 2002.
Dove et al., Nature, 390 (6656): 187-192, 1997.
Elbashir et al., Nature, 411 (6836): 494-498, 2001.
EPO 0 273 085
Fechheimer et al., Proc. Natl. Acad. Sci. USA, 84: 8463-8467, 1987.
Ferkol et al., FASEB J, 7: 1081-1091, 1993.
Fire et al., Nature, 391: 806-811, 1998.
Forster and Symons, Cell, 49 (2): 211-220, 1987.
Foti et al., Mol. Biol. Cell, 12 (8): 2396-411, 2001.
Fraley et al., Proc. Natl. Acad. Sci. USA, 76: 3348-3352, 1979.
Freifelder, In: Physical Biochemistry Applications to Biochemistry and Molecular Biology, 2Ed. Wm. Freeman and Co., NY, 1982.
Friedmann, Science, 244: 1275-1281, 1989.
Gary et al., J. Cell Biol, 143 (1): 65-79, 1998.
Gerlach et al., Nature, 328: 802-805, 1987.
Ghosh and Bachhawat, In: Liver diseases, targeted diagnosis and therapy using specific receptors and ligands, Wu and Wu (Eds.), NY, Marcel Dekker, 87-104, 1991.
Ghosh-Choudhury et al., EMBO J, 6: 1733-1739, 1987.
Gomez-Foix et al., J. Biol. Chem., 267: 25129-25134, 1992.
Gopal, Mol. Cell Biol., 5: 1188-1190, 1985.
Graham and Prevec, Biotechnology, 20: 363-390, 1992.
Graham and Prevec, In: Methods in Molecular Biology: Gene Transfer and Expression Protocol, Murray (Ed.), Clifton, N.J., Humana Press, 7: 109-128, 1991.
Graham and van der Eb, Virology, 52: 456-467, 1973.
Graham et al., J. Gen. Virol., 36: 59-72, 1977.
Grishok et al., Science, 287: 2494-2497, 2000.
Grunhaus and Horwitz, Seminar in Virology, 3: 237-252, 1992.
Guo et al., Bioorg. Med. Chem. Lett., 9: 419, 1999.
Guo et al., J. Biol. Chem., 274: 12990-995, 1999.
Hardie et al., Neuron, 30 (1): 149-159, 2001.
Harland and Weintraub, J. Cell Biol., 101: 1094-1099, 1985.
Hawkins et al., Biochem J, 238 (2): 507-516, 1986.
Hermonat and Muzyczka, Proc. Natl. Acad. Sci. USA, 81: 6466-6470, 1984.
Hersdorffer et al., DNA Cell Biol., 9: 713-723, 1990.
Herz and Gerard, Proc. Natl. Acad. Sci. USA, 90: 2812-2816, 1993.
Hogan et al., In: Manipulating the Mouse Embryo: A Laboratory Manual, 2nd ed., Cold Spring Harbor Laboratory Press, 1994.
Horwich et al. J. Virol., 64: 642-650, 1990.
Huang et al., Nature, 391 (6669): 803-806, 1998.
Hughes et al., J. Biol. Chem., 275 (2): 801-8, 2000.
Huijbregts et al., Traffic, 1 (3): 195-202, 2000.
Ikonomov et al., J. Biol. Chem., 2:2, 2001.
Innis et al., PCR Protocols, Academic Press, Inc., San Diego Calif., 1990.
Itoh et al., J. Biol. Chem., 275 (25): 19389-19394, 2000.
Janez et al., Endocrinology, 141 (12): 4657-4663, 2000a.
Janez et al., J. Biol. Chem., 275 (35): 26870-26876, 2000b.
Johnson et al., In: Biotechnology and Pharmacy, Pezzuto et al., Eds., Chapman and Hall, New York, 1993.
Joly et al., J. Biol. Chem., 270 (22): 13225-13230, 1995.
Jones and Shenk, Cell, 13: 181-188, 1978.
Jones et al., J. Biol., 274 (26): 18407-18413, 1999.
Joyce, Nature, 338: 217-244, 1989.
Kaneda et al., Science, 243: 375-378, 1989.
Karlsson et al., EMBO J, 5: 2377-2385, 1986.
Kato et al., J. Biol. Chem., 266: 3361-3364, 1991.
Ketting et al., Cell, 99: 133-141, 1999.
Kim and Cook, Proc. Natl. Acad. Sci. USA, 84 (24): 8788-8792, 1987.
Klein et al., Nature, 327: 70-73, 1987.
Kyte and Doolittle, J Mol Biol, 157 (1): 105-32, 1982.
Lanier and Gertler, Curr. Biol., 10 (18): R655-657, 2000.
Le Gal La Salle et al., Science, 259: 988-990, 1993.
Levrero et al., Gene, 101: 195-202, 1991.
Li et al., J. Biol. Chem., 276 (25): 22883-22891, 2001.
Lin et al., Nature, 402: 128-129, 1999.
Macejak and Sarnow, Nature, 353: 90-94, 1991.
Maehama and Dixon, J. Biol. Chem., 273 (22): 13375-13378, 1998.
Mann et al., Cell, 33: 153-159, 1983.
Markowitz et al., J. Virol., 62: 1120-1124, 1988.
Marshall et al., J. Biol. Chem., 271: 183, 1996.
McEwen et al., J. Biol. Chem., 274 (48): 33905-33912, 1999.
Merrifield, Science, 232: 341-347, 1986.
Michel and Westhof, J. Mol. Biol., 216: 585-610, 1990.
Montgomery et al., Proc. Natl. Acad. Sci. USA, 95: 155-2-15507, 1998.
Nicolas and Rubinstein, In: Vectors: A survey of molecular cloning vectors and their uses, Rodriguez and Denhardt (Eds.), Stoneham, Butterworth, 494-513, 1988.
Nicolau and Sene, Biochim. Biophys. Acta, 721: 185-190, 1982.
Nicolau et al., Methods Enzymol., 149: 157-176, 1987.
Palmiter et al., Nature, 300: 611, 1982.
Paskind et al., Virology, 67: 242-248, 1975.
Pattni et al., Curr. Biol., 11 (20): 1636-1642, 2001.
PCT App. WO 00/44914
PCT App. WO 01/36646
PCT App. WO 01/68836
PCT App. WO 90/07641
PCT App. WO 99/32619
Pelletier and Sonenberg, Nature, 334: 320-325, 1988.
Perales et al., Proc. Natl. Acad. Sci. USA, 91: 4086-4090, 1994.
Pignon et al., Hum. Mutat., 3: 126-132, 1994.
Potter et al., Proc. Natl. Acad. Sci. USA, 81: 7161-7165, 1984.
Racher et al., Biotech. Tech., 9: 169-174, 1995.
Ragot et al., Nature, 361: 647-650, 1993.
Reggiori and Pelham, Nat. Cell Biol., 4: 117-23, 2002.
Reinhold-Hurek and Shub, Nature, 357: 173-176, 1992.
Renan, Radiother. Oncol., 19: 197-218, 1990.
Rich et al., Hum. Gene Ther., 4: 461-476, 1993.
Ridgeway, In: A survey of molecular cloning vectors and their uses, Rodriguez et al. (Eds.), Stoneham, Butterworth, 467-492, 1988.
Rippe et al., Mol. Cell Biol., 10: 689-695, 1990.
Rosenfeld et al., Cell, 68: 143-155, 1992.
Rosenfeld et al., Science, 252: 431-434, 1991.
Roux et al., Proc. Natl. Acad. Sci. USA, 86: 9079-9083, 1989.
Sambrook et al., “Molecular Cloning,” A Laboratory Manual, 2d Ed., Cold Spring Harbor Laboratory Press, New York, 13.7-13.9: 1989.
Sarver et al., Science, 247: 1222-1225, 1990.
Sbrissa et al., Biochemistry, 39 (51): 15980-15989, 2000.
Sbrissa et al., J. Biol. Chem., 274 (31): 21589-21597, 1999.
Scanlon et al., Proc. Natl. Acad. Sci. USA, 88: 10591-10595, 1991.
Schafer, Curr. Opin. Cell Biol., 14 (1): 76-81, 2002.
Schu et al., Science, 260 (5104): 88-91, 1993.
Sharp et al., Science, 287: 2431-2433, 2000.
Sharp Genes Dev., 13: 139-141, 1999.
Sherman, Methods Enzymol., 194: 3-21, 1991.
Shisheva et al., J. Biol. Chem., 276 (15): 11859-11869, 2001.
Shisheva et al., Mol. Cell Biol., 19 (1): 623-634, 1999.
Sikorski and Hieter, Genetics, 122 (1): 19-27, 1989.
Simonsen et al., Curr. Opin. Cell Biol., 13 (4): 485-492, 2001.
Srinivasan et al., Eur. J. Cell Biol., 74 (4): 350-60, 1997.
Stack et al., Annu. Rev. Cell Dev. Biol., 11: 1-33, 1995.
Stewart and Young, In: Solid Phase Peptide Synthesis, 2Ed., Pierce Chemical Co., 1984.
Stolz et al., J. Biol. Chem., 273 (19): 11852-61, 1998a.
Stolz et al., Genetics, 148 (4): 1715-29, 1998b.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene Ther., 1: 241-256, 1990.
Sui et al., Proc. Natl. Acad. Sci. USA, 95 (3): 1307-1312, 1998.
Tabara et al., Cell, 99: 123-132, 1999.
Tam et al., J. Am. Chem. Soc., 105: 6442, 1983.
Taylor et al., Proc. Natl. Acad. Sci. USA, 97 (16): 8910-8915, 2000.
Temin, In: Gene Transfer, Kucherlapati (Ed.), NY, Plenum Press, 149-188, 1986.
Toker and Cantley, Nature, 387 (6634): 673-676, 1997.
Top et al., J. Infect. Dis., 124: 155-160, 1971.
Tur-Kaspa et al., Mol. Cell Biol., 6: 716-718, 1986.
Urbanowski et al., Traffic, 2: 622-39, 2001.
Vancurova et al. J. Biol. Chem., 274 (2): 1147-1155, 1999.
Varmus et al., Cell, 25: 23-36, 1981.
Vida and Emr, J. Cell Biol., 128 (5): 779-792, 1995.
Vieira et al., J. Cell Biol., 155 (1): 19-25, 2001.
Wagner et al., Science, 260: 1510-1513, 1990.
Wang et al., J. Cell Biol., 140 (5): 1063-1074, 1998.
Wang et al., Science, 274, 784-787, 1996.
Weisman et al., J. Cell Biol., 105: 1539-1547, 1987
Whiteford et al., Biochem J, 319 (Pt3): 851-860, 1996.
Whiteford et al., Biochem. J, 323 (Pt3): 597-601, 1997.
Wincott et al., Nucleic Acids Res., 23 (14): 2677-2684, 1995.
Wong et al., Gene, 10: 87-94, 1980.
Wu and Wu, Adv. Drug Delivery Rev., 12: 159-167, 1993.
Wu and Wu, Biochemistry, 27: 887-892, 1988.
Wu and Wu, J. Biol. Chem., 262: 4429-4432, 1987.
Yang et al., J. Virol., 68: 4847-4856, 1994a.
Yang et al., Proc. Natl. Acad. Sci. USA, 87: 9568-9572, 1990.
Zelenin et al., FEBS Lett., 280: 94-96, 1991.

1. An isolated and purified nucleic acid encoding a polypeptide comprising the sequence of SEQ ID NO:1 (Fab1 Human AA).

2. The isolated and purified nucleic acid of claim 1, wherein the nucleic acid comprises the sequence of SEQ ID NO:2 (Fab1 Human DNA).

3. An isolated and purified nucleic acid encoding a polypeptide comprising the sequence of SEQ ID NO:3 (Vac14 Human AA).

4. The isolated and purified nucleic acid of claim 3, wherein the nucleic acid comprises the sequence of SEQ ID NO:4 (Vac14 Human DNA).

5. An isolated and purified nucleic acid encoding a polypeptide comprising the sequence of SEQ ID NO:5 (Fig4 Human AA).

6. The isolated and purified nucleic acid of claim 1, wherein the nucleic acid comprises the sequence of SEQ ID NO:6 (Fig4 Human DNA).

7. An isolated and purified nucleic acid encoding a polypeptide comprising the sequence of SEQ ID NO:7 (Vac14 yeast AA).

8. The isolated and purified nucleic acid of claim 1, wherein the nucleic acid comprises the sequence of SEQ ID NO:8 (Vac14 yeast DNA).

9. An expression construct comprising a promoter active in eukaryotic cells, said promoter operably linked to a nucleic acid segment encoding a polypeptide comprising the sequence of SEQ ID NO:1, SEQ ID NO:3, SEQ ID NO:5 or SEQ ID NO:7.

10. An oligonucleotide of between about 10 and about 50 bases, said oligonucleotide comprising at least about 10 consecutive bases of SEQ ID NO:1, SEQ ID NO:3, SEQ ID NO:5 or SEQ ID NO:7.

11. The oligonucleotide of claim 10, wherein said oliogonucleotide is 10, 15, 20, 25, 30, 35, 40, 45 or 50 bases in length.

12. The oligonucleotide of claim 10, wherein the number of consecutive bases is 10, 15, 20, 25, 30, 35, 40, 45 or 50 bases.

13. A recombinant cell comprising an expression cassette comprising a promoter active in eukaryotic cells, said promoter operably linked to a nucleic acid segment encoding a polypeptide comprising the sequence of SEQ ID NO:2, SEQ ID NO:4, SEQ ID NO:6 or SEQ ID NO:8, said promoter also being heterologous to said nucleic acid segment.

14. The recombinant cell of claim 13, wherein said expression cassette is comprised in an episomal element.

15. The recombinant cell of claim 13, wherein said expression cassette is integrated into the cellular genome.

16. An isolated and purified polypeptide comprising the sequence of SEQ ID NO:1 (Fab1 Human AA).

17. The isolated and purified polypeptide of claim 16, wherein said polypeptide is a fusion protein further comprising additional non-human Fab1 sequences.

18. An isolated and purified polypeptide comprising the sequence of SEQ ID NO:3 (Vac14 Human AA).

19. The isolated and purified polypeptide of claim 18, wherein said polypeptide is a fusion protein further comprising additional non-human Vac14 sequences.

20. An isolated and purified polypeptide comprising the sequence of SEQ ID NO:5 (Fig4 Human AA).

21. The isolated and purified polypeptide of claim 20, wherein said polypeptide is a fusion protein further comprising additional non-Fig4 sequences.

22. An isolated and purified polypeptide comprising the sequence of SEQ ID NO:7 (Vac14 yeast AA).

23. The isolated and purified polypeptide of claim 22, wherein said polypeptide is a fusion protein further comprising additional non-yeast Vac14 sequences.

24. An oligopeptide of between about 5 and about 30 residues, said oligopeptide comprising at least about 5 consecutive residues of SEQ ID NO:1, SEQ ID NO:3, SEQ ID NO:5 or SEQ ID NO:7.

25. The oligopeptide of claim 24, wherein said oliogopeptide is 5, 10, 15, 20, 25, or 30 residues in length.

26. The oligopeptide of claim 24, wherein the number of consecutive residues is 5, 10, 15, 20, 25, or 30.

27. A monoclonal antibody that binds immunologically to a polypeptide comprising the sequence of SEQ ID NO:1 (Fab1 Human AA), the sequence of SEQ ID NO:3 (Vac14 Human AA), the sequence of SEQ ID NO:5 (Fig4 Human AA), or the sequence of SEQ ID NO:7 (Vac14 yeast AA).

28. A polyclonal antisera, antibodies of which bind immunologically to a polypeptide comprising the sequence of SEQ ID NO:1 (Fab1 Human AA), the sequence of SEQ ID NO:3 (Vac14 Human AA), the sequence of SEQ ID NO:5 (Fig4 Human AA), or the sequence of SEQ ID NO:7 (Vac14 yeast AA).

29. A non-human transgenic animal, cells of which comprise expression cassette comprising a promoter active in eukaryotic cells, said promoter operably linked to a nucleic acid segment encoding a polypeptide comprising the sequence of SEQ ID NO:1, SEQ ID NO:3, SEQ ID NO:5 or SEQ ID NO:7, said promoter also being heterologous to said nucleic acid segment.

30. A non-human transgenic animal, cells of which exhibit a defect in the expression of a Fab1, Vac14 or Fig4 polypeptide.

31. The non-human transgenic animal of claim 30, wherein said cells exhibit reduced expression of Fab1, Vac14 or Fig4 polypeptide.

32. The non-human transgenic animal of claim 30, wherein said cells exhibit expression of a reduced-function or non-functional Fab1, Vac14 or Fig4 polypeptide.

33. A method of identifying a subject at risk of developing diabetes comprising assessing the structure, function or expression of Fab1, Vac14 and/or Fig4 in cells of said subject.

34. The method of claim 33, wherein assessing comprises assessing expression.

35. The method of claim 34, wherein assessing expression comprises Northern blotting.

36. The method of claim 34, wherein assessing expression comprises quantitative RT-PCR.

37. The method of claim 34, wherein assessing expression comprises Western blotting.

38. The method of claim 34, wherein assessing expression comprises quantitative immunohistochemistry.

39. The method of claim 33, wherein assessing comprises assessing activity.

40. The method of claim 39, wherein assessing activity comprises measuring PI(3,5)P₂.

41. The method of claim 40, wherein assessing activity comprises measuring PI(3,5)P₂ turnover.

42. The method of claim 40, wherein assessing activity comprises measuring PI(3,5)P₂ steady state levels.

43. The method of claim 40, wherein assessing activity comprises measuring PI(3,5)P₂ synthesis.

44. The method of claim 40, wherein assessing activity comprises measuring PI(3)P.

45. The method of claim 39, wherein assessing activity comprises measuring protein kinase activity.

46. The method of claim 33, wherein assessing comprises assessing structure.

47. The method of claim 46, wherein assessing structure comprises nucleic acid sequencing.

48. The method of claim 47, wherein sequence comprises PCR.

49. The method of claim 47, wherein sequence comprises RT-PCR.

50. The method of claim 49, wherein assessing structure comprises measuring antibody binding.

51. The method of claim 50, wherein measuring antibody binding comprises, RIA, ELISA, Western blot or immunohistochemistry.

52. The method of claim 46, wherein assessing structure comprises high stringency nucleic acid hybridization.

53. The method of claim 33, further comprising obtaining a cell from said subject.

54. The method of claim 53, wherein said cell is a kidney cell, a liver cell, a leukocyte, an adipocyte, or a muscle cell.

55. The method of claim 53, further comprising subjecting said cell to stress prior to assessing expression or activity.

56. The method of claim 55, wherein stress is osmotic stress.

57. The method of claim 55, further comprising subjecting said cell to hormonal stimulation prior to assessing expression or activity.

58. The method of claim 57, wherein said hormonal stimulation is insulin stimulation.

59. A method of screening a candidate compound for their ability to increase glucose uptake comprising:
(a) providing a insulin-responsive cell;
(b) contacting said insulin-responsive cells with said candidate compound; and
(c) measuring the change in PI(3,5)P₂ in said cell.

60. The method of claim 49, wherein said insulin-responsive cell is an adipocyte or a muscle cell.
